SEQUENCE LISTING (3 sequences)

SEQ ID NO: 1 - protein, 14 aa, Artificial
  Synthetic TKD sequence
TKDNNLLGREFLSG

SEQ ID NO: 2 - protein, 8 aa, Artificial
  Synthetic Aptamer A8
SPWPRPTY

SEQ ID NO: 3 - protein, 13 aa, Artificial
  Synthetic Aptamer A17
YCAYYSPRHKTTF

(19) United States
(12) Patent Application Publication
(10) Pub. No.: US 2017/0131285 A1
(43) Pub. Date: May 11, 2017

(54) METHODS AND COMPOSITIONS FOR DIAGNOSING, MONITORING AND TREATING CANCER

(71) Applicants: INSERM (Institut National de la Sante et de la Recherche Medicale), Paris (FR); Universite de Bourgogne, Dijon (FR)

(72) Inventors: Carmen Garrido-Fleury, Dijon (FR); Jessica Gobbo, Dijon (FR); Renaud Seigneuric, Dijon (FR); Guillaume Marcion, Dijon (FR)

(21) Appl. No.: 15/318,185

(22) PCT Filed: Jun. 12, 2015
(86) PCT No.: PCT/EP2015/063186

§ 371 Date: Dec. 12, 2016

(30) Foreign Application Priority Data
Jun. 13, 2014 (EP) 14305898.0

(51) Int. Cl. G01N 33/574; A61K 38/08

(52) U.S. Cl. CPC G01N 33/57488; A61K 38/08; G01N 2333/47

(57) ABSTRACT

The invention relates to methods for diagnosing cancer and determining the responsiveness to chemotherapyin a subject based on the detection of HSP70-expressing exosomes in a bodily fluid sample obtained from said subject. The invention also relates to methods for treating cancer and in particular methods for restoring or enhancing the anti-tumor immune response in a patient in need thereof by inhibiting the activation MDSC as well as methods for inhibiting or reducing HSP70-expressing exosomes-mediated tumor resistance against a chemotherapeutic agent and therefore methods for restoring or enhancing the efficacy of said chemotherapeutic agent.

### FIELD OF THE INVENTION

The invention relates to the field of oncology. The invention provides methods for diagnosing cancer and determining the responsiveness to chemotherapy based on the detection of HSP70-expressing exosomes in a bodily fluid sample obtained from a subject. The invention also provides new combination therapy and methods for treating cancer and in particular methods for restoring or enhancing the anti-tumor immune response in a patient in need thereof by inhibiting the activation MDSC as well as methods for inhibiting or reducing HSP70-expressing exosomes-mediated tumor resistance against a chemotherapeutic agent and therefore methods for restoring or enhancing the efficacy of said chemotherapeutic agent.

### BACKGROUND OF THE INVENTION

Diagnostic and surgical resection together with conventional anticancer drugs have highly improved survival in patients with cancer [1-3]. Unfortunately, morbidity and mortality cancer rates remain high [3,4]. Currently, a growing body of evidences suggest that the success of an anticancer therapy depends on the anti-cancer immune response efficiency [5,6]. Recent studies have identified myeloid cells as potent suppressors of tumor immunity and, therefore represent a major limitation for cancer immunotherapy [7]. These cells, named myeloid-derived suppressor cells (MDSC), accumulate in the blood, spleen, lymph nodes, bone marrow and at tumor sites in most patients and in experimental models. MDSC plays a major role in inhibition of both adaptive and innate immunity [8]. MDSC are considered as essential actors in the immune dysfunction observed in most patients with sizable tumor burdens [9]. Accordingly, the presence of MDSC in cancer patients is correlated with poor survival and tumor progression [10,11]. How MDSC affect the immune system is still unclear but likely act via the suppression of lymphocytes and NK cells activity. MDSC are activated by host- and tumor-secreted factors and Chalmin et al. have recently demonstrated that tumor-derived exosomes (TDE), through membrane-anchored HSP70, can also activate MDSC [12].

Exosomes are small nanovesicles (˜50-200 nm in diameter) released into the extracellular environment from cells via the endosomal vesicle pathway by fusion with the plasma membrane [13,14]. A broad range of cells secrete exosomes including T/B cells [15,16], epithelial [17] and dendritic cells [18] as well as tumor cells. These exosomes are essential for intercellular communication [19]. TDE have been described to play a major role in the formation of primary tumors and metastases [20] and to modulate anti-tumor immune responses [21]. Most importantly, MDSC activation depends on the expression of the heat shock protein 70 (HSP70) present at the surface of these TDE [12,22]. Indeed, MDSC activation results from the interaction between toll-like receptor 2 (TLR2), present on their surface and HSP70, expressed by exosomes. This interaction leads to the stimulation of NF-κB (Nuclear Factor-kappa-B) signaling pathway and then, activation of the signaling pathway JAK2 (Janus Kinase)/STAT3 through IL-6 autocrine secretion. HSP70 is a stress-inducible heat shock protein with intra- and extracellular functions (danger signal role). Intracellular roles of HSP70 include the chaperone function through the stabilization of protein 3D-structures, prevention of protein aggregation by binding to unfolded proteins and, anti-apoptotic functions [23,24]. HSP70 is overexpressed in many cancer cells and confers resistance to chemotherapeutic drugs promoting cancer development. Cancer cells, because they have to re-wire their metabolism, have a strong need for chaperones like HSP70. Accordingly, the down-regulation of HSP70 is sufficient to kill or sensitize tumor cells to apoptosis induction in vitro and can impair tumorigenicity in vivo [25,26].

HSP70 can also be found at the plasma membrane of cancer cells but not normal cells. Indeed, in contrast to cancer cells, normal cells hardly express HSP70 and since only about 10% of the total amount of intracellular HSP70 is re-located in the plasma membrane, that explains the absence of detectable membrane-anchored HSP70 [27]. Membrane-bound HSP70 has been shown to have immune modulatory functions including favoring the resistance of cancer cells to immune-mediated destruction [28,29]. Membrane-bound HSP70 presents an extracellular sequence composed of 14 amino acids in the C-terminus region (TKD sequence), against which a monoclonal antibody has been recently raised (cmHSP70) [30]. Injection of this antibody cmHSP70 into mice bearing CT26 tumors, significantly inhibited tumor growth and enhanced the overall survival. These effects were associated with infiltrations of natural killer (NK) cells, macrophages, and granulocytes [31].

### SUMMARY OF THE INVENTION

In a first aspect, the invention relates to an in vitro method for diagnosing cancer in a subject, comprising a step of determining the level of Heat Shock Protein 70 (HSP70)-expressing exosomes in a bodily fluid sample obtained from said subject.

In a second aspect, the invention relates to an in vitro method for determining the responsiveness of a patient to a treatment with a chemotherapeutic agent, comprising a step of determining the level of HSP70-expressing exosomes in a bodily fluid sample obtained from said patient.

In a third aspect, the invention relates to an inhibitor of the interaction between HSP70 and Toll-like receptor 2 (TLR2) for use in a method for restoring or enhancing the anti-tumor immune response in a patient in need thereof by inhibiting the activation of myeloid-derived suppressor cells (MDSC), wherein said inhibitor is a HSP70 binding agent that specifically binds to an epitope of HSP70 that is localized extracellularly on said exosomes.

In a fourth aspect, the invention relates to an inhibitor of the interaction between HSP70 and TLR2 for use in a method for inhibiting or reducing HSP70-expressing exosomes-mediated tumor resistance against a chemotherapeutic agent, wherein said inhibitor is a HSP70-binding agent that specifically binds to an epitope of HSP70 that is localized extracellularly on said exosomes.

In a fifth aspect, the invention relates to an inhibitor of the interaction between HSP70 and TLR2 for use in a method for restoring or enhancing the efficacy of a chemotherapeutic agent, wherein said inhibitor is a HSP70-binding agent that specifically binds to an epitope of HSP70 that is localized extracellularly on said exosomes.

In a last aspect, the invention relates to an in vitro method for determining whether a patient will benefit from a treatment with an inhibitor of the interaction between HSP70 and TLR2 such as a peptide of SEQ ID NO: 2, comprising a step of determining the level of HSP70-expressing exosomes in a bodily fluid sample obtained from said patient.

### DETAILED DESCRIPTION OF THE INVENTION

Exosomes, via heat shock protein-70 (HSP70) expressed in their membrane, are able to interact with the toll-like receptor 2 (TLR2) on myeloid derived suppressive cells (MDSC) thereby activating them. Here, the inventors showed that A8 peptide aptamer binds to the extracellular domain of membrane HSP70 and set up a protocol using A8 to capture HSP70-exosomes from cancer patients' samples. They demonstrated that the number of HSP70-exosomes is much higher in cancer patients than in healthy donors. Accordingly, all cancer cell lines examined released HSP70-exosomes while “normal” cells do not. HSP70 has higher affinity for A8 than for TLR2. Therefore, A8 blocks HSP70/TLR2 association and the ability of tumor-derived exosomes to activate MDSC. Treatment of tumor-bearing mice with A8 induces a decrease in the number of MDSC in the spleen and inhibits tumor progression. A chemotherapeutic agent like cisplatin increases the amount of HSP70-exosomes favoring the activation of MDSC and hampering the development of an anti-tumor immune response. In contrast, this MDSC activation is not observed if cisplatin is combined with A8. As a result, the anti-tumor effect of cisplatin is strongly potentiated. In conclusion, A8 is useful to quantify tumor-derived exosomes and for cancer therapy through MDSC inhibition.

### Diagnostic Methods of the Invention

In a first aspect, the invention relates to an in vitro method for diagnosing cancer in a subject, comprising a step of determining the level of Heat Shock Protein 70 (HSP70)-expressing exosomes in a bodily fluid sample obtained from said subject.

As used herein, the term “diagnosing” includes determining whether a subject suffers or not from a disease such as cancer, predicting whether a subject is at risk of suffering from a disease such as cancer, determining the likelihood of recovery from a disease such as cancer, and predicting the probable course and/or outcome of a disease such as cancer including predicting whether a subject is at risk for cancer recurrence or cancer resistance.

As used herein, the term “cancer” refers to or describes the pathophysiological condition in mammals (including humans) that is typically characterized by unregulated cell growth.

In one embodiment of the invention, the cancer is selected from the group consisting of lung, colon, breast, ovary head and neck, stomach, prostate, cervix, pancreas carcinomas, malignant melanoma, hematological diseases, and lymphoma.

As used herein, the term “determining” includes qualitative and/or quantitative detection (i.e. detecting and/or measuring the level) with or without reference to a control or a predetermined value. As used herein, “detecting” means determining if HSP70-expressing exosomes are present or not in a biological sample and “measuring” means determining the level of HSP70-expressing exosomes in a biological sample.

As used herein, the term “HSP70” (also known as HSP72) refers to the Heat Shock 70 kDa Protein which is a protein of 641 amino acids that in humans is encoded by the HSPA1A gene. This intronless gene encodes a 70 kDa heat shock protein (HSP) which is a member of the heat shock protein 70 family. This protein stabilizes existing proteins against aggregation and mediates the folding of newly translated proteins in the cytosol and in organelles. The naturally occurring human HSP70 gene has a nucleotide sequence as shown in Genbank Accession number NM_005345.5 and the naturally occurring human HSP70 protein has an amino acid sequence as shown in Genbank Accession number NP_005336.3.

As used herein, the term “exosome” refers to vesicles (typically about 30-150 nm) secreted by cells by a mechanism of exocytosis of internal vesicles. Recently, it was shown that tumor cells are capable of secreting vesicles. These vesicles usually correspond to an internal vesicle contained in an endosome of a tumor cell and secreted by said tumor cell subsequent to the fusion of the external membrane of said endosome with cytoplasmic membrane of above-mentioned tumor cell.

As used herein, the terms “cancer cell” or “tumor cell” are used interchangeably and refer to the total population of cells derived from a tumor or a pre-cancerous lesion.

As used herein, the terms “HSP70-exosomes” or “HSP70-expressing exosomes” refer to tumor-derived exosomes expressing HSP70 on their membrane.

As used herein, the term “bodily fluid sample” has its general meaning in the art and refers to any bodily fluid sample which may be obtained from a subject for the purpose of in vitro evaluation. A preferred body fluid sample is a blood sample (e.g. whole blood sample, serum sample, or plasma sample) or a urine sample.

As used herein, the term “subject” has its general meaning in the art and refers to a mammal, such as a rodent, a feline, a canine, and a primate. Preferably, a subject according to the invention is a human.

The step of determining the presence and/or the level of HSP70-expressing exosomes may comprise a step of contacting the biological sample with selective reagents such as antibodies or aptamers, and thereby detecting the presence, or measuring the level, of exosomes of interest originally in said biological sample. Contacting may be performed in any suitable device, such as a plate, microtiter dish, test tube, well, glass, column, and so forth.

In one embodiment, the methods according to the invention comprise a step of contacting the biological sample with a binding partner capable of selectively interacting with HSP70-expressing exosomes in said biological sample.

Thus, the step of determining the level of HSP70-expressing exosomes in a bodily fluid sample obtained from said subject comprises the steps of:

(a) contacting the bodily fluid sample with a HSP70-binding agent that binds to an extracellular localized epitope of membrane-bound HSP70 on said exosomes in order to isolate HSP70-expressing exosomes, and

(b) determining the level of HSP70-expressing exosomes thus isolated.

For instance, membrane-bound HSP70 presents an extracellular sequence composed of 14 amino acids in the C-terminus region (TKDNNLLGRFELSG or TKD sequence as defined in SEQ ID NO: 1), against which a monoclonal antibody has been recently raised (cmHSP70) disclosed in the European Patent EP1706423B1.

| Name | Sequence | Length (aa) | SEQ ID NO: |
|---|---|---|---|
| TKD peptide | TKDNNLLGREFLSG | 14 | 1 |

In one embodiment of the invention, said HSP70-binding agent is an agent (such as an aptamer or an antibody) which specifically binds to an epitope of HSP70 that is localized extracellularly (it is intended that the term “extracellularly” refers to exosomes and therefore is interchangeably used with the term “extramembranously” and corresponds to “an extramembranous epitope” preferably located in the C-terminal region of HSP70 present on exosomal membrane) on said exosome.

In another embodiment of the invention, said HSP70-binding agent is a peptide of SEQ ID NO: 2 (called herein “Aptamer A8”) or a function-conservative variant as defined in the Section entitled “Therapeutic Uses” below. It should be further noted that Aptamer A8 binds to an epitope of HSP70 that is localized extracellularly on said exosome such as the TKD sequence of SEQ ID NO: 1.

| Name | Sequence | Length (aa) | SEQ ID NO: |
|---|---|---|---|
| Aptamer A8 | SPWPRPTY | 8 | 2 |

As used herein, the term “peptide” refers to an amino acid sequence having less than 15 amino acids, preferably less than 10 amino acids. The term also applies to amino acid polymers in which one or more amino acid residue is an artificial chemical mimetic of a corresponding naturally occurring amino acid, as well as to naturally occurring amino acid polymers and non-naturally occurring amino acid polymer.

In another embodiment, the step of determining the level of HSP70-expressing exosomes in a bodily fluid sample obtained from said subject comprises the steps of:

(a) isolating the exosomes contained in said bodily fluid sample,

(b) lysing the exosomes thus obtained resulting in disruption of said exosomes, and

(c) determining the level expression of HSP70 protein.

The step of exosome isolation may be carried out by methods well-known in the art such as centrifugation, ultracentrifugation, filtration or ultrafiltration. Exosomes may be further fractionated using conventional methods such as ultracentrifugation with or without the use of a density gradient to obtain higher purity. Exosomes may also be isolated from other micro-vesicules by using other properties of the exosome such as the presence of surface markers. Surface markers which may be used include Flotilin-1 and CD81. As an example, exosomes having CD81 on their surface may be isolated using antibody coated magnetic particles. Dynabeads® are super-paramagnetic polystyrene beads which may be conjugated with anti-human CD81 antibody, either directly to the bead surface or via a secondary linker (e.g. anti-mouse IgG).

The step of exosome lysis may be carried out by using any standard lysis protocol comprising detergents such Triton-X-100, NP-40 or RIPA buffer.

Once the exosomes are lysed, the expression level of HSP70 may be determined by any known method in the art including standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis and immunoprecipitation.

In a particular embodiment, such methods comprise a step of contacting the sample containing the lysed exosomes with a binding partner capable of selectively interacting with HSP70 present in said sample. In one embodiment, the binding partner may be an antibody that may be polyclonal or monoclonal, preferably monoclonal.

In another embodiment, the binding partner may be an aptamer such as the peptide aptamer A8 described above or a function-conservative variant thereof as defined in the Section entitled “Therapeutic Uses” below or any peptide aptamers already described for instance peptide aptamer A17 of SEQ ID NO: 3 which are known for binding HSP70.

| Name | Sequence | Length (aa) | SEQ ID NO: |
|---|---|---|---|
| Aptamer A17 | YCAYYSPRHKTTF | 13 | 3 |

The binding partners of the invention such as antibodies or aptamers, useful in the different embodiments as described above, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody or aptamer, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited to radioactive atoms for scintigraphic studies such as I¹²³, I¹²⁴, In¹¹¹, Re¹⁸⁶, Re¹⁸⁸ as well as conjugated DOTA derivatives wherein DOTA is 1,4,7,10-tetraazacyclododecane-1,4,7,10-tetraacetic acid such as ¹¹¹In-DOTA-aptamer or ⁶⁸Ga-DOTA-aptamer.

The peptides of the invention may be produced by any suitable means, as will be apparent to those of skill in the art. In order to produce sufficient amounts of peptide aptamer A8 or functional equivalents thereof, for use in accordance with the invention, expression may conveniently be achieved by culturing under appropriate conditions recombinant host cells containing the peptide of the invention. Preferably, the peptide is produced by recombinant means, by expression from an encoding nucleic acid molecule. Systems for cloning and expression of a peptide in a variety of different host cells are well known.

In one embodiment of the invention, the step of comparing said level of HSP70-expressing exosomes with reference values obtained from the same subject or healthy subjects wherein detecting a difference in the level of HSP70-expressing exosomes with the reference values is indicative whether the subject has, or is at risk of having cancer.

In a particular embodiment, the invention relates to an in vitro method for diagnosing cancer in a subject, comprising the followings steps of:

(a) determining the level of HSP70-expressing exosomes in a bodily fluid sample obtained from said patient,

(b) comparing said level of HSP70-expressing exosomes with reference values, wherein a increase in the level of HSP70-expressing exosomes with the reference values is indicative whether the subject has, or is at risk of having cancer.

The method of the invention also allows the detection of a resistance to a chemotherapeutic agent as described below. A high level of HSP70-expressing exosomes is predictive of a non-response to chemotherapy.

As used herein, the terms “resistance” or “resistant” refer to a lack of response by a cell to an agent to which the cell may have responded previously (e.g. the cell is “resistant to” such agent). In the context of a subject, “resistance” refers to lack of response of a subject to an agent to which said subject used to respond. Resistance can be acquired (e.g. develops over time) or inherent or de novo (e.g. a cell or subject never responds to an agent to which other similar cells or subjects would respond). By way of non-limiting example, a subject is said to be resistant to treatment when such subject no longer responds to such treatment (e.g. the treatment of a subject with an agent results in initial delay of disease progression, but then such disease progresses even if said subject is still treated with such agent.)

In a second aspect, the invention relates to an in vitro method for determining the responsiveness of a patient to chemotherapy, comprising a step of determining the level of HSP70-expressing exosomes in a bodily fluid sample obtained from said patient.

As used herein, the term “responsiveness” refers to the development of a favorable response when a cell or subject is contacted with an agent (e.g. a therapeutic agent.) By way of non-limiting example, a favorable response can be inhibition of cell growth when a cell is contacted with a particular agent and an unfavorable response can be the accelerated growth of a tumor when a patient with a tumor is contacted with a particular agent.

As used herein, the term “determining the responsiveness of a patient to a chemotherapy” should be understood broadly, and accordingly encompasses a determination made before starting any treatment with a chemotherapeutic agent (i.e. predicting the responsiveness of a patient to a chemotherapeutic agent and determination made during a treatment with a chemotherapeutic agent (i.e. monitoring the responsiveness of a patient to a chemotherapeutic agent).

As used herein, the term “chemotherapy” is the treatment of cancer with one or more cytotoxic anti-neoplastic drugs (chemotherapeutic agents) as part of a standardized regimen

As used herein, the term “chemotherapeutic agent” refers to a compound that elicits a response from a cell or patient when said cell or patient is contacted with said compound. A chemotherapeutic agent can be a small molecule, a peptide, an antibody, a natural product, a nucleic acid, etc. as described below. The term “chemotherapeutic agent” is a broad one covering many chemotherapeutic agents having different mechanisms of action.

For instance, the chemotherapeutic agent is selected from the group consisting of a microtubule active agent, an alkylating agent, an anti-neoplastic anti-metabolite, a platin compound, a topoisomerase I inhibitor, a topoisomerase II inhibitor, a VEGF inhibitor, an insulin-like growth factor I inhibitor, a protein tyrosine kinase inhibitor, an EGFR kinase inhibitor, a mTOR kinase inhibitor, a proteasome inhibitor, a HDAC inhibitor, a PI3K/AKT inhibitor, a RAF kinase inhibitor and tumor cell damaging approaches, such as ionizing radiation.

The term “microtubule active agent,” as used herein, relates to microtubule stabilizing, microtubule destabilizing agents and microtublin polymerization inhibitors including, but not limited to taxanes, e.g., paclitaxel and docetaxel; vinca alkaloids, e.g., vinblastine, especially vinblastine sulfate; vincristine, especially vincristine sulfate and vinorelbine; discodermolides; colchicine and epothilones and derivatives thereof, e.g., epothilone B or a derivative thereof. Paclitaxel is marketed as taxol; docetaxel as taxotere; vinblastine sulfate as vinblastin and vincristine sulfate as farmistin.

The term “alkylating agent” as used herein, includes, but is not limited to, Dacarbazine (DTIC) cyclophosphamide, ifosfamide, melphalan or nitrosourea (BCNU or Gliadel), or temozolomide (temodar). Cyclophosphamide can be administered, e.g., in the form as it is marketed, e.g., under the trademark cyclostin; and ifosfamide as holoxan.

The term “anti-neoplastic anti-metabolite” as used herein, includes, but is not limited to, 5-fluorouracil (5-FU); capecitabine; gemcitabine; DNA de-methylating agents, such as 5-azacytidine and decitabine; methotrexate; edatrexate; and folic acid antagonists. Capecitabine can be administered, e.g., in the form as it is marketed, e.g., under the trademark xeloda; and gemcitabine as gemzar.

The term “platin compound” as used herein, includes, but is not limited to, carboplatin, cisplatin, cisplatinum, oxaliplatin, satraplatin and platinum agents, such as zd0473. Carboplatin can be administered, e.g., in the form as it is marketed, e.g., carboplat; and oxaliplatin as eloxatin. In the field of oncology the platin compound are often designated as alkylating-like compounds.

The term “topoisomerase I inhibitor” as used herein, includes derivatives of the plant compound camptothecin. Irinotecan (CPT-11) is a semi-synthetic derivative of camptothecin. Topotecan is another semi-synthetic analogue of camptothecin. There are other derivatives of camptothecin, as well as new formulations of the parent plant extract, that are in various stages of clinical trials.

The term “topoisomerase II inhibitor” as used herein, includes, but is not limited to, the anthracyclines, such as doxorubicin, including liposomal formulation, e.g., caelyx; daunorubicin, including liposomal formulation, e.g., daunosome; epirubicin; idarubicin and nemorubicin; the anthraquinones mitoxantrone and losoxantrone; and the podophillotoxines etoposide and teniposide. Etoposide is marketed as etopophos; teniposide as VM 26-bristo; doxorubicin as adriblastin or adriamycin; epirubicin as farmorubicin; idarubicin as zavedos; and mitoxantrone as novantron.

The term “VEGF inhibitor”, as used herein, includes compounds targeting, decreasing or inhibiting the activity of the vascular endothelial growth factor (VEGF) receptors, such as compounds that target, decrease or inhibit the activity of VEGF, especially compounds that inhibit the VEGF receptor, such as, but not limited to, 7/−/−pyrrolo[2,3-d]pyrimidine derivative; BAY 43-9006; isolcholine compounds disclosed in WO 00/09495, such as (4-tert-butyl-phenyl)-94-pyridin-4-ylmethyl-isoquinolin-1-yl)-amine.

The term “insulin-like growth factor I inhibitor”, as used herein, relates to compounds targeting, decreasing or inhibiting the activity of the insulin-like growth factor receptor 1 (IGF-1 R), such as compounds that target, decrease or inhibit the activity of IGF-IR, especially compounds that inhibit the IGF-1 R receptor. Compounds include, but are not limited to, the compounds disclosed in WO02/092599 and derivatives thereof of 4-amino-5-phenyl-7-cyclobutyl-pyrrolo {2,3-pyrimidine derivatives.

The term “protein tyrosine kinase inhibitor”, as used herein, relates to compounds targeting, decreasing or inhibiting the activity of protein-tyrosine kinase, such as imatinib mesylate (gleevec), tyrphostin orpyrymidylaminobenzamide and derivatives thereof. A tyrphostin is preferably a low molecular weight (M<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556; AG957; and adaphostin (4-{[(2,5-dihydroxyphenyl)methyl]amino}-benzoic acid adamantyl ester).

The term “EGFR kinase inhibitor” as used herein, relates to compounds targeting, decreasing or inhibiting the activity of the epidermal growth factor family of receptor tyrosine kinases (EGFR, ErbB2, ErbB3, ErbB4 as homo- or heterodimers), such as compounds that target, decrease or inhibit the activity of the epidermal growth factor receptor family are especially compounds, proteins or antibodies that inhibit members of the EGF receptor tyrosine kinase family, e.g., EGF receptor, ErbB2, ErbB3 and ErbB4 or bind to EGF or EGF-related ligands, and are in particular those compounds, proteins or monoclonal antibodies generically and specifically disclosed in WO97/02266, e.g., the compound of Example 39, or in EP0564409, WO99/03854, EP0520722, EP0566226, EP0787722, EP0837063, U.S. Pat. No. 5,747,498, WO98/10767, WO97/30034, WO97/49688, WO97/38983 and WO 96/30347, e.g., compound known as CP358774, WO96/33980, e.g., compound ZD1839; and WO95/03283, e.g., compound ZM105180, e.g., trastuzumab (HERCEPTIN), cetuximab, iressa, OSI-774, CI-1033, EKB-569, GW-2016, E1.1, E2.4, E2.5, E6.2-E6.4, E2.11, E6.3 or E7.6.3, and erlotinib and gefitinib. Erlotinib can be administered in the form as it is marketed, e.g., tarceva, and gefitinib as iressa, human monoclonal antibodies against the epidermal growth factor receptor including ABX-EGFR.

The term “mTOR kinase inhibitor”, as used herein, refers to compounds that target, decrease or inhibit the activity/function of serine/theronine mTOR kinase are especially compounds, proteins or antibodies that target/inhibit members of the mTOR kinase family, e.g., RAD, RAD001, CCI-779, ABT578, SAR543, rapamycin and derivatives/analogs thereof, AP23573 and AP23841 from Ariad, everolimus (certican) and sirolimus.

The term “proteasome inhibitor” as used herein, refers to compounds that target, decrease or inhibit the activity/function of the proteosome. Compounds that target, decrease or inhibit the activity of the proteosome include, but are not limited to, PS-341; MLN 341, bortezomib or velcade.

The term “HDAC inhibitor,” as used herein, relates to compounds that inhibit the histone deacetylase and that possess anti-proliferative activity. This includes, but is not limited to, compounds disclosed in WO 02/22577. It further especially includes suberoylanilide hydroxamic acid (SAHA); [4-(2-amino-phenylcarbamoyl)-benzyl]carbamic acid pyridine-3-ylmethyl ester and derivatives thereof; butyric acid, pyroxamide, trichostatin A, oxamflatin, apicidin, depsipeptide, depudecin and trapoxin.

The term “PI3K/AKT inhibitor,” as used herein, refers to compounds that target, decrease or inhibit the phosphatidylinositol 3-kinase (PI3K)/Akt pathway. The latter pathway is considered as a critical survival-signaling pathway. Akt-mediated phosphorylation may alter the activity of proteins such as caspase-9, some Bcl-2 family members, and nuclear factor [kappa]B (NF-[kappa]B) and other transcription factors, which trigger or restrain apoptosis; and PI3K/Akt deregulation may contribute to tumorigenesis, metastasis, and resistance to chemotherapy. Inhibition of Akt activation and activity can be achieved by inhibiting PI3K with inhibitors such as LY294002 and wortmannin In a particular embodiment a PI3/AKT kinase inhibitor is an AKT inhibitor. Akt, alternatively named as protein kinase B, is a serine/threonine kinase.

The term “RAF kinase inhibitor” refers to compounds that interfere with the abnormal activation of a RAF kinase. The meaning of “abnormal activation of a RAF kinase” is further explained. The Ras/Raf/Mek/ERK (mitogen-activated protein kinase) signaling pathway plays a critical role in transmitting proliferation signals generated by the cell surface receptors and cytoplasmic signaling elements to the nucleus. Constitutive activation of this pathway is involved in malignant transformation by several oncogenes. Activating mutations in RAS occur in approximately 15% of cancers, and recent data has shown that the RAF kinase, B-RAF, is mutated in about 7% of cancers (Wellbrock et al., Nature Rev. Mol. Cell. Biol. 2004, 5:875-885). In mammals, the RAF family of serine/threonine kinases comprises three members: A-RAF, B-RAF and C-RAF. However, activating mutations have so far been only identified in B-RAF underlining the importance of this isoform.

The term “tumor cell damaging approaches” refers to approaches, such as ionizing radiation. The term “ionizing radiation,” referred to above and hereinafter, means ionizing radiation that occurs as either electromagnetic rays, such as X-rays and gamma rays; or particles, such as alpha, beta and gamma particles. Ionizing radiation is provided in, but not limited to, radiation therapy and is known in the art. See Hellman, Cancer, 4th Edition, Vol. 1, Devita et al., Eds., pp. 248-275 (1993).

As used herein, the term “patient” refers to any subject (preferably human) afflicted with a disease likely to benefit from a treatment with a chemotherapeutic agent. Said disease is preferably cancer. In one embodiment of the invention, cancer is selected from the group consisting of lung, colon, breast, ovary head and neck, stomach, pancreas carcinomas, malignant melanoma, and hematological diseases.

The method of the invention may further comprise a step of comparing the level of HSP70-expressing exosomes with reference values obtained from responder and non-responder group of patients, wherein detecting a difference in the level of HSP70-expressing exosomes with the reference values is indicative whether the patient will be or is a responder or not to the treatment with a chemotherapeutic agent.

A “responder” patient refers to a patient who shows a clinically significant relief in the disease when treated with a chemotherapeutic agent.

In a particular embodiment, the invention relates to an in vitro method for determining the responsiveness of a patient to chemotherapy, comprising the followings steps of:

(a) determining the level of HSP70-expressing exosomes in a bodily fluid sample obtained from said patient,

(b) administering to said patient a therapeutically amount of a chemotherapeutic agent,

(c) determining the level of HSP70-expressing exosomes in a bodily fluid sample obtained from said treated patient,

(d) comparing the levels determined at step (a) and step (c), wherein a decrease between said levels is indicative of a response to said chemotherapeutic agent.

After being tested for responsiveness to a treatment with a chemotherapeutic agent, the patients may be prescribed with said chemotherapeutic agent or if the chemotherapy already started, the chemotherapy may be continued.

Alternatively, the patients may be prescribed with said chemotherapeutic agent in combination with an inhibitor of the interaction between HSP70 and Toll-like receptor 2 (TLR2) such as the aptamer A8 as described below or if the chemotherapy already started, the chemotherapy may be continued in combination with said inhibitor.

Accordingly, in another aspect of the invention, the invention relates to an in vitro method for determining whether a patient will benefit from a treatment with an inhibitor of the interaction between HSP70 and TLR2, preferably a peptide of SEQ ID NO: 2 (called herein “Aptamer A8”) or a function-conservative variant, comprising a step of determining the level of Heat Shock Protein 70 (HSP70)-expressing exosomes in a bodily fluid sample obtained from said patient.

### Kits of the Invention

Yet another aspect of the invention relates to a kit for performing a method of the invention, said kit comprising means for determining the level of HSP70-expressing exosomes in a bodily fluid sample obtained from a subject. The kit may include the peptide aptamer A8, or a set of peptides aptamers A8 and A17 as above described. In a particular embodiment, the aptamer or set of aptamers are labelled as above described. The kit may also contain other suitably packaged reagents and materials needed for the particular detection protocol, including solid-phase matrices, if applicable, and standards. The kit may also contain one or more means for the detection of exosomes such as anti-CD81 antibodies.

A further aspect of the invention relates to the use of HSP70-expressing exosomes as a biomarker for diagnosing cancer in a subject or for determining the responsiveness of a patient to chemotherapy. The invention also relates to the use of a kit of the invention for diagnosing cancer in a subject or for determining the responsiveness of a patient to chemotherapy. The invention relates to the use of a kit comprising means for determining the level of HSP70-expressing exosomes in a bodily fluid sample obtained from a subject for performing a method for diagnosing cancer in a subject or a method for determining the responsiveness of a patient to chemotherapy.

### Therapeutic Methods and Uses

The invention also relates to an inhibitor of the interaction between HSP70 and TLR2, preferably a peptide of SEQ ID NO: 2 or a function-conservative variant method for treating a patient in need thereof, comprising the following steps of
(a) identifying whether a patient is responsive to chemotherapy with the method for determining the responsiveness of the invention; and
(b) treating with an inhibitor of the interaction between HSP70 and TLR2 the non-responder patient identified.

As used herein, the term “treating” a disease or a condition refers to reversing, alleviating or inhibiting the process of one or more symptoms of such disorder or condition.

In one embodiment of the invention, said HSP70-binding agent is a peptide of SEQ ID NO: 2 (called herein “Aptamer A8”) or a function-conservative variant as defined above.

In one embodiment of the invention, said chemotherapeutic agent is selected from the group consisting of a microtubule active agent, an anti-neoplastic anti-metabolite, a platin compound and a topoisomerase II inhibitor as defined above.

Another aspect of the invention relates to a method for treating with an inhibitor of the interaction between HSP70 and TLR2, preferably a peptide of SEQ ID NO: 2 or a function-conservative variant a patient in need thereof, comprising the following steps of
(a) identifying whether a patient is responsive to chemotherapy with the method for determining the responsiveness of the invention; and
(b) treating with an inhibitor of the interaction between HSP70 and TLR2 the non-responder patient identified.

The invention also provides methods and compositions (such as pharmaceutical and kit-of part compositions) for use in restoring or enhancing the anti-tumor immune response in a patient in need thereof by inhibiting the activation of myeloid-derived suppressor cells (MDSC). The invention also provides methods and compositions for use in inhibiting or reducing HSP70-expressing exosomes-mediated tumor resistance against a chemotherapeutic agent. The invention further provides methods and compositions for use in increasing the efficacy of a chemotherapeutic agent.

In a third aspect, the invention relates to an inhibitor of the interaction between HSP70 and Toll-like receptor 2 (TLR2) for use in a method for restoring or enhancing the anti-tumor immune response in a patient in need thereof by inhibiting the activation of MDSC, wherein said inhibitor is a HSP70 binding agent that specifically binds to an epitope of HSP70 that is localized extracellularly on said exosomes.

As used herein, the term “restoring or enhancing the anti-tumor immune response” refers to the ability of a particular substance to provoke an immune response. Restoring or enhancing tumor immunogenicity aids in the clearance of the tumor cells by the immune response. As described herein, HSP70-expressing exosomes following administration of said chemotherapeutic agent activate myeloid-derived suppressor cells (MDSC).

The terms “inhibiting the interaction” or “inhibitor of the interaction” are used herein to mean preventing or reducing the direct or indirect association of one or more molecules, peptides, proteins, enzymes or receptors; or preventing or reducing the normal activity of one or more molecules, peptides, proteins, enzymes, or receptors. The term “inhibitor of the interaction between HSP70 and TLR2” is a molecule which can prevent the interaction between HSP70 and TLR2 by competition or by fixing to one of the molecule.

As used herein, the term “Toll-like receptor 2” (TLR2) refers to a protein that in humans is encoded by the TLR2 gene. TLR2 has also been designated as CD282 (cluster of differentiation 282). TLR2 is one of the toll-like receptors and plays a role in the immune system. The naturally occurring human TLR2 gene has a nucleotide sequence as shown in Genbank Accession number NM_003264.3 and the naturally occurring human TLR2 protein has an aminoacid sequence as shown in Genbank Accession number NP_036035.3.

In one embodiment of the invention, said HSP70-binding agent is a peptide of SEQ ID NO: 2 (called herein “Aptamer A8”) or a function-conservative variant.

| Name | Sequence | Length (aa) | SEQ ID NO: |
|---|---|---|---|
| Aptamer A8 | SPWPRPTY | 8 | 2 |

As used herein, the term “function-conservative variant” refers to a peptide in which a given amino acid residue in peptide has been changed (inserted, deleted or substituted) without altering the overall conformation and function of the peptide. Such variants include peptides having amino acid alterations such as deletions, insertions and/or substitutions. A “deletion” refers to the absence of one or more amino acids in the protein. An “insertion” refers to the addition of one or more of amino acids in the protein. A “substitution” refers to the replacement of one or more amino acids by another amino acid residue in the protein. Typically, a given amino acid is replaced by an amino acid having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 80% to 99% as determined according to an alignment scheme such as by the Cluster Method, wherein similarity is based on the MEGALIGN algorithm.

A “function-conservative variant” also includes a peptide having at least about 80% amino acid sequence identity with the native sequence polypeptide. Such variants include, for instance, polypeptides wherein one or more amino acid residues are added, or deleted, at the N- or C-terminus of the peptide. Ordinarily, a variant will have at least about 80% amino acid sequence identity, more preferably at least about 90% amino acid sequence identity, and even more preferably at least about 95% amino acid sequence identity with the peptide of interest.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid. In the frame of the present application, the percentage of identity is calculated using a global alignment (i.e., the two sequences are compared over their entire length). Methods for comparing the identity and homology of two or more sequences are well known in the art. The “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Peptides consisting of an amino acid sequence “at least 80%, 85%, 90%, 95%, 96%, 97%, 98% or 99% identical” to a reference sequence may comprise mutations such as deletions, insertions and/or substitutions compared to the reference sequence. The peptide consisting of an amino acid sequence at least 80%, 85%, 90%, 95%, 96%, 97%, 98% or 99% identical to a reference sequence may comprise substitutions compared to the reference sequence. The substitutions preferably correspond to conservative substitutions as indicated in the table below.

| Conservative substitutions | Type of Amino Acid |
|---|---|
| Ala, Val, Leu, lle, Met, | Amino acids with aliphatic |
| Pro, Phe, Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

A “function-conservative variant” refers to a peptide exhibiting at least one, preferably all, of the biological activities of the reference peptide such as the peptide aptamer A8, provided the function-conservative variant retains the capacity of binding to an extracellular localized epitope of membrane-bound HSP70 on HSP70-expressing exosomes. Additionally, the function-conservative variant may further be characterized in that it is capable of inhibiting the interaction between HSP70 and TLR2 (see Example); and/or inhibiting the activation of myeloid-derived suprresor cells (MDSC) (see Example).

In one embodiment, the function-conservative variant of the invention may comprise chemical modifications improving their stability and/or their biodisponibility. Such chemical modifications aim at obtaining peptides with increased protection against enzymatic degradation in vivo, and/or increased capacity to cross membrane barriers, thus increasing its half-life and maintaining or improving its biological activity. Any chemical modification known in the art can be employed according to the present invention.

Such chemical modifications include but are not limited to:
replacement(s) of an amino acid with a modified and/or unusual amino acid, e.g. a replacement of an amino acid with an unusual amino acid like Nle, Nva or Orn; and/or modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the β carbon rather than the α carbon.

In a fourth aspect, the invention relates to an inhibitor of the interaction between HSP70 and TLR2 for use in a method for inhibiting or reducing HSP70-expressing exosomes-mediated tumor resistance against a chemotherapeutic agent, wherein said inhibitor is a HSP70-binding agent that specifically binds to an epitope of HSP70 that is localized extracellularly on said exosomes.

In this context, the term “exosomes-mediated tumor resistance” is to be understood as a mechanism of a cell to escape the action and/or effect of a chemotherapeutic agent by formation of HSP70-expressing exosomes following administration of said chemotherapeutic agent to a patient in need thereof, which will therefore activate myeloid-derived suppressor cells (MDSC). As a consequence, MDSC inhibit the antitumor immune response of said chemotherapeutic agent independently of its mechanisms of action.

In one embodiment of the invention, said HSP70-binding agent is a peptide of SEQ ID NO: 2 (called herein “Aptamer A8”) or a function-conservative variant as defined above.

In one embodiment of the invention, said chemotherapeutic agent is selected from the group consisting of a microtubule active agent, an anti-neoplastic anti-metabolite, a platin compound and a topoisomerase II inhibitor as defined above.

In a fifth aspect, the invention relates to an inhibitor of the interaction between HSP70 and TLR2 for use in a method for restoring or enhancing the efficacy of a chemotherapeutic agent, wherein said inhibitor is a HSP70-binding agent that specifically binds to an epitope of HSP70 that is localized extracellularly on said exosomes.

As used herein, the term “restoring or enhancing the efficacy” refers to the increase of the number of patients affected with a cancer and treated with a chemotherapeutic compound which exhibit a clinically beneficial response to said treatment.

As used herein, the “patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

Preferably, said beneficial response is a long-term response. Accordingly, the term “long-term response” is used herein to refer to a complete response for at least 1 year, more preferably for at least 3 years, most preferably for at least 5 years following treatment.

In one embodiment of the invention, said HSP70-binding agent is a peptide of SEQ ID NO: 2 (called herein “Aptamer A8”) or a function-conservative variant as defined above.

In one embodiment of the invention, said chemotherapeutic agent is selected from the group consisting of a microtubule active agent, an anti-neoplastic anti-metabolite, a platin compound and a topoisomerase II inhibitor as defined above.

The present invention also relates to a method for restoring or enhancing the anti-tumor immune response in a patient in need thereof by inhibiting the activation of myeloid-derived suppressor cells (MDSC), comprising the step of administering to said subject an inhibitor of the interaction between HSP70 and Toll-like receptor 2 (TLR2) wherein said inhibitor is a HSP70 binding agent that specifically binds to an epitope of HSP70 that is localized extracellularly on said exosomes.

The present invention also relates to a method for inhibiting or reducing HSP70-expressing exosomes-mediated tumor resistance against a chemotherapeutic agent, comprising the step of administering to said subject an inhibitor of the interaction between HSP70 and TLR2 wherein said inhibitor is a HSP70-binding agent that specifically binds to an epitope of HSP70 that is localized extracellularly on said exosomes.

The present invention also relates to a method for restoring or enhancing the efficacy of a chemotherapeutic agent, comprising the step of administering to said subject an inhibitor of the interaction between HSP70 and TLR2 wherein said inhibitor is a HSP70-binding agent that specifically binds to an epitope of HSP70 that is localized extracellularly on said exosomes.

### Pharmaceutical Compositions

In another aspect, the invention relates to a pharmaceutical composition comprising an inhibitor of the interaction between HSP70 and TLR2 as above-defined and a chemotherapeutic agent.

Pharmaceutical compositions comprising an inhibitor of the interaction between HSP70 and TLR2 of the invention include all compositions wherein said inhibitor is contained in an amount effective to achieve the intended purpose. In addition, the pharmaceutical compositions may contain suitable physiologically acceptable carriers comprising excipients and auxiliaries which facilitate processing of the active compounds into preparations which can be used pharmaceutically.

The term “physiologically acceptable carrier” is meant to encompass any carrier, which does not interfere with the effectiveness of the biological activity of the active ingredient and that is not toxic to the host to which is administered. Suitable physiologically acceptable carriers are well known in the art and are described for example in Remington's Pharmaceutical Sciences (Mack Publishing Company, Easton, USA, 1985), which is a standard reference text in this field. For example, for parenteral administration, the above active ingredients may be formulated in unit dosage form for injection in vehicles such as saline, dextrose solution, serum albumin and Ringer's solution.

In one embodiment, the inhibitor of the interaction between HSP70 and TLR2 such as the peptide of SEQ ID NO: 2 (Aptamer A8) is administered in association with a delivery system including but not limited to liposomes, dendrimers, and microencapsulation.

In another embodiment, the inhibitor of the interaction between HSP70 and TLR2 such as the peptide of SEQ ID NO: 2 (Aptamer A8) is coated on a nanoparticle. Thus, the nanoparticle displays at its surface the inhibitor according to the invention.

Within the context of the invention, the nanoparticles are of small size, small enough to be taken up by cells to allow HSP70 inhibition. In preferred embodiments, the nanoparticles have a mean diameter between 0.1 and 300 nm.

The core of the nanoparticle may be a polymeric core. Preferably, the nanoparticle comprises polymers are selected from the group consisting of carbohydrate-based polymers (e.g., cellulose-based nanoparticles, chitosan-based nanoparticles), polyethylene glycol (PEG), polypropylene glycol (PPG), and copolymers of PEG and PPG, branched copolymers containing PEG and caprolactone, PEG and lactide, and PEG and [lactide-co-glycolide].

The core of the nanoparticle may also be a metallic core. Preferably, the metallic core comprises Au, Ag or Cu, for example an alloy selected from Au/Ag, Au/Cu, Au/Ag/Cu, Au/Pt, Au/Pd, Au/Ag/Cu/Pd, Au/Fe, Au/Cu, Au/Gd, Au/Fe/Cu, Au/Fe/Gd or Au/Fe/Cu/Gd.

Preferably, the nanoparticles are soluble in most organic solvents and especially water. Nanoparticles can be prepared according to techniques well-known in the art.

Besides the physiologically acceptable carrier, the pharmaceutical compositions of the invention can also comprise minor amounts of additives, such as stabilizers, excipients, buffers and preservatives. The pharmaceutical composition of the invention may further comprise a chemotherapeutic agent.

In one embodiment of the invention, said HSP70-binding agent is a peptide of SEQ ID NO: 2 (called herein “Aptamer A8”) or a function-conservative variant as defined above.

Combinations of chemotherapeutic agents with HSP70-binding agent such as aptamer A8 result in synergistic effects and in improvements in cancer therapy. Generally, chemotherapeutic agents are classified according to the mechanism of action. Many of the available agents are anti-metabolites of development pathways of various tumors, or react with the DNA of the tumor cells as above-defined.

In one embodiment of the invention, said chemotherapeutic agent is selected from the group consisting of a microtubule active agent, an anti-neoplastic anti-metabolite, a platin compound and a topoisomerase II inhibitor as defined above.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intraocular, intravenous, intramuscular or subcutaneous administration.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

To prepare pharmaceutical compositions, an effective amount of peptide of the invention may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The peptides according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In another aspect, the invention relates to a pharmaceutical composition comprising an inhibitor of the interaction between HSP70 and TLR2 as above-defined for use in a method for restoring or enhancing the anti-tumor immune response in a patient in need thereof by inhibiting the activation of MDSC.

In another aspect, the invention relates to a pharmaceutical composition comprising an inhibitor of the interaction between HSP70 and TLR2 as above-defined for use in a method for inhibiting or reducing HSP70-expressing exosomes-mediated tumor resistance against a chemotherapeutic agent.

In another aspect, the invention relates to a pharmaceutical composition comprising an inhibitor of the interaction between HSP70 and TLR2 as above-defined for use in a method for restoring or enhancing the efficacy of a chemotherapeutic agent.

In another aspect, the invention relates to a method for restoring or enhancing the anti-tumor immune response in a patient in need thereof by inhibiting the activation of MDSC comprising the followings steps of (i) administering a therapeutically effective amount of a chemotherapeutic agent as above-defined, and (ii) administering a therapeutically effective amount of an inhibitor of the interaction between HSP70 and TLR2 as above-defined, wherein step (i) is conducted before, concomitant or after to step (ii).

In another aspect, the invention relates to a method for reducing exosomes-mediated tumor resistance against a chemotherapeutic agent, comprising the steps of (i) administering a therapeutically effective amount of a chemotherapeutic agent, and (ii) administering a therapeutically effective amount of an inhibitor of the interaction between HSP70 and TLR2 as above-defined, wherein step (i) is conducted before, concomitant or after to step (ii).

In another aspect, the invention relates to a method of restoring or enhancing the efficacy of a chemotherapeutic agent, comprising the steps of (i) administering a therapeutically effective amount of a chemotherapeutic agent, and (ii) administering a therapeutically effective amount of an inhibitor of the interaction between HSP70 and TLR2 as above-defined, wherein step (i) is conducted before, concomitant or after to step (ii).

As used herein, the term “therapeutically effective amount” is intended for a minimal amount of active agent, which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient such as a cancer.

### Kit-of-Parts Compositions

The inhibitor of the interaction between HSP70 and TLR2 of the invention such as the aptamer A8 and the chemotherapeutic agent may be combined within one formulation and administered simultaneously. However, they may also be administered separately, using separate compositions. It is further noted that they may be administered at different times.

The invention relates to a kit-of-parts composition comprising an inhibitor of the interaction between HSP70 and TLR2 as above-defined and a chemotherapeutic agent.

The invention relates to a kit-of-parts composition comprising an inhibitor of the interaction between HSP70 and TLR2 as above-defined and a chemotherapeutic agent for use in a method for inhibiting or reducing HSP70-expressing exosomes-mediated tumor resistance against a chemotherapeutic agent.

The invention relates to a kit-of-parts composition comprising an inhibitor of the interaction between HSP70 and TLR2 as above-defined and a chemotherapeutic agent for use in a method for increasing the efficacy of said chemotherapeutic agent.

The terms “kit”, “product” or “combined preparation”, as used herein, define especially a “kit-of-parts” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes. When the administration is sequential, the first partner may be for instance administered 1, 2, 3, 4, 5, 6, 7, days before the second partner.

The invention will be further illustrated by the following figures and examples. However, these examples and figures should not be interpreted in any way as limiting the scope of the present invention.

### FIGURES

FIG. 1: Tumor-derived exosomes in cancer patient′ samples express HSP70. (A) The binding of exosomes (10⁶), derived from the B16F10, HCT116 cell lines, with or without previous incubation with cmHSP70, to immobilized biotinylated A8 was determined by biolayer interferometry. (B) Association curves of HSP70-exosomes in patients' urines (1 breast, 1 pulmonary) or healthy individuals (controls) with biotinylated A8 immobilized on streptavidin sensor tips. Curves represent mean values of triplicate measurements for each concentration. (C) Exosomes expressing HSP70 were determined by FACS in urine samples from healthy individuals (n=5) and cancer metastatic cancer patients (5 breast, 4 pulmonary). The bar graph presents the means and standard deviations of the vesicle concentrations. (D) Nanoparticle Tracking Analysis (NTA) to quantify total number of exosomes in urine human samples (urines) described above. Samples were diluted in PBS and analyzed using a NanoSight LM10 instrument. Inset values represent the total number of exosomes counted 10̂⁹/ml.

FIG. 2: Exosomes derived from human and mice cancer cell lines express HSP70 on their membrane. (A) A representative western blot of Flotillin-1 and CD81 expression in exosomes isolated from MEF, B16F10, CT26, HCT116 and SW480 cells' supernatants. (B) A representative transmission electron microscopy image of exosomes derived from B16F10, CT26, HCT116 and SW480 cancer cell lines as well as MEF, immunogold labeling using cmHSP70-FITC antibody (10 nm of gold). Scale bar, 100 nm. An arrow indicates the presence of HSP70. (C) Binding of exosomes (10⁶), derived from MEF, B16F10, CT26, HCT116 and SW480 cells, to immobilized biotinylated cmHSP70 antibody was determined by biolayer interferometry. MEF that showed little binding are considered as a negative control for HSP70-exosomes. (D) Membrane-bound-HSP70 mean fluorescence intensity (MFI) in exosomes isolated from B16F10, CT26, HCT116, SW480 and MEF was determined by flow cytometry. Data represent MFI+/−SD (n=3); insert shows representative FACS histogram. *, p<0.05.

FIG. 3: A8 blocks HSP70/TLR2 association. (A) Immunoprecipitation of TLR2 in the presence of increasing concentrations of A8 (from 0.3 μM to 1.8 μM) was followed by HSP70 immunoblotting. IP control, no relevant IgG antibody. (B) Denatured luciferase (D) was incubated in the absence or presence of human HSP70 and, when indicated, increasing concentrations of A8 (C1=5 μM; C2=15 μM; C3=25 μM and C4=50 μM) for 1 hour at 25° C. Following incubation, the luciferase substrate D-luciferin was added and within 10 minutes the total light units emitted were collected for 10 seconds at 560 nm using a Wallac (Victor 3) spectrophotometer. *, p<0.05. (C) Biolayer interferometry to determine association and dissociation curves of HSP70 (concentration range from 2.5 μM to 156 nM) with biotinylated TRL2 immobilized on streptavidin sensor tips. (D) Association and dissociation curves of HSP70 (concentration range from 50 nM to 1.28 nM) with biotinylated A8 immobilized on streptavidin sensor tips. Curves in C and D represent mean values of triplicate measurements for each concentration.

FIG. 4: A8 blocks the activation and proliferation of MSC2 cells. (A) IL-6 concentration was determined by ELISA in the supernatant of MSC2 cells incubated or not for 24 h with exosomes (from B16F10) in the presence or absence of A8. *, p<0.05, (n=3). (B) A representative image of a western blot showing the kinetics of STAT3 phosphorylation in MCS2 cells incubated or not with exosomes isolated from B16F10, CT26 or HCT116 cells together with a peptide control (TDE) or A8 (TDE/A8). 14-3-3 is used as a loading control. In the right panels, the amount of pSTAT3 was quantified by densitometric analysis. *, p<0.05, n=4.

FIG. 5: A8 anti-tumor effect in mice is associated to MDSC and an immune anti-tumor response. (A) Mice were sc injected with B16F10 cells (5×10⁴). On day 4, when tumor size was about 0.9 mm³, animals were treated every two days until the end of the experiment with A0 or A8 (3 mg/kg dissolved in PBS, i.p injection). On day 5, half the animals were treated with cisplatin as a single dose (CDDP, i.p injection, 5 mg/kg). At day 18, the percentage of Gr1⁺ CD11b⁺ cells in the spleen collected from the different groups of mice was analyzed by flow cytometry (6 animals per group). As a MDCS negative control (Ctl) we used animals with no tumor cells injected. Right panels, a representative immunoblot of pSTAT3 in the MDSC (Gr1⁺ CD11b⁺ cells) isolated from the spleen of control animals (A0/PBS) or A8 and CDDP-treated animals (A8/CDDP). 14-3-3 is used as a loading control. (B) Analysis of tumor growth in the animals described above. Tumor size was measured every two days (6 animals per group). The white symbols represent the tumors from animals treated with cisplatin. (C) HSP70-exosomes isolated from B16F10 cells treated or not with cisplatin (CDDP, 25 μM) were determined by flow cytometry. Data represent the percentage of MFI+/−SD (n=3); *, p<0.05. (D) Association curves of HSP70-exosomes present in tumor-bearing mice sera treated or not with cisplatin (5 mg/kg). For the biolayer interferometry, biotinylated A8 was immobilized on streptavidin sensor tips. Curves represent mean values of triplicate measurements for each concentration (for each curve, the blood of 5 mice per group was pooled).

FIG. 6: Exosomes derived from human and mice cancer cell lines express HSP70 on their membrane. Binding of exosomes (10⁶), derived from PC3, HeLa, MCF-7 and EL4 cancer cells, to immobilized biotinylated A8 was determined by biolayer interferometry. Each cancer cell was compared to the following normal counterparts: PrEC (Prostate Epithelial Cells), PUC (Primary Uterine Cells), HMEC (Human Mammary Epithelial Cells) and PL (Primary Lymphocytes).

FIG. 7: Effect of chemotherapy drugs on HSP70-exosomes release. A) Association curves of HSP70-exosomes isolated from the supernatant of HCT116 cells treated or not with oxaliplatin (5 μM), 5FU (3 μM), paclitaxel (50 nM), gemcitabin (10 μM) and doxorubicin (2.5 mM). For the BLI, biotinylated A8 was immobilized on streptavidin sensor tips, n=3. B-C) HCT116 cells were treated with 5FU (3, 12, 24 and 36 μM) or oxaliplatin (5, 20, 40 and 60 μM) or doxorubicin (2.5, 10, and 20 μM) during 48 h. B) concentration of HSP70-exosomes released was measured by flow cytometry. C) measured apoptosis of HCT116 (7AAD and FITC-Annexin V).

FIG. 8: A8 blocks the activation of primary MDSC cells. A) A representative image of a western blot showing the kinetics of STAT3 phosphorylation in MDSC cells incubated or not with exosomes isolated from B16F10 cells together with a peptide control (MDSC/TDE) or A8 (MDSC/TDE/A8). B-C) IL-6 (B) and IL-10 (C) concentration was determined by ELISA in the supernatant of MDSC cells incubated or not for 24 h with exosomes (from B16F10) in the absence or presence of A8 (16 μM). D) IFNγ concentration was determined by ELISA in the supernatant of co-cultured MDSC and T lymphocytes incubated or not for 5 days with exosomes (from B16F10) in the absence or presence of A8 (1604). **, p<0.01; *, p<0.1 (n=3).

FIG. 9: TDE and A8 effects on MSC2 and MDSC proliferation. A) Dynamic monitoring of in vitro MSC2 cell proliferation using the RTCA-DP system (Real-Time Cell Analyzer). MSC2 were incubated or not for 11 h with exosomes (from B16F10) in the absence or presence of A8 (16 μM and 32 μM). Cell index values were measured every 30 seconds (n=3). B) MSC2 were incubated or not for 24 h with exosomes (from B16F10) in the absence or presence of A8 (16 μM). Then, cells were labelled with BrdU and incorporation was determined by flow cytometry (n=3). C) MDSC were treated with exosomes (from B16F10) in the absence or presence of A8 (1604) for 24 hours and then analyzed for proliferation by Cellometer-mini. *p<0.05.

FIG. 10: A8 anti-tumor effect in mice is associated with an increased anti-tumor immune response. Mice were s.c. injected with EL4 cells (7·10⁵). On day 5, animals were treated every two days until the end of the experiment with either A0 (control aptamer) or A8 (3 mg/kg, i.p. injection). On day 6, half the animals were i.p. treated with a single dose of 5-fluorouracil (5FU, 25 mg/kg). A) At day 10, the percentage of MDSC (Gr1⁺ CD11b⁺) cells in the spleen collected from the different groups of mice was analyzed by flow cytometry (6 animals per group). As a negative control (Ctl) we used animals with no tumors. B) Tumor size was measured every two days (6 animals per group; n=3). The open symbols represent animals treated with 5FU. T test: *, p<0.05, **, p<0.01, (n=3). C) EL4 tumor sections were performed 10 days after injection of A8. Dendritic cells and macrophages were labeled using, CD11c and F4/80 antibodies. A representative image is shown (n=6 per group). Labeled cells were counted from 300 cells chosen randomly in different microscopic fields.

### EXAMPLES

### Example 1: Restoring Anticancer Immune Response by Targeting Tumor-Derived Exosomes with a HSP70 Peptide Aptamer

Material & Methods

Cells and Products:

B16F10 mouse melanoma, CT26 human colon cancer, HCT116 and SW480 human colorectal cancer cell lines (American Type Culture Collection) were cultured in RPMI 10% foetal bovine serum (FBS, Lonza) Mouse embryonic fibroblasts (MEF) were cultured in DMEM 10% FBS (Lonza). MSC2 cell line (myeloid suppressor cell 2), a gift from V. Bronte (Istituto Oncologico, Padova, Italy), was cultured in RPMI 10% FBS. Absence of mycoplasma contamination was assayed every week.

The peptide aptamer A8 of SED ID NO: 2 (SPWPRPTY) and A17 of SEQ ID NO: 3 (YCAYYSPRHKTTF) from the variable region of the aptamers were synthesized and highly purified by Proteogenix. They were reconstituted and diluted in PBS at the indicated concentrations. Recombinant HSPs were from StressGen (EnzoLife). Recombinant TLR2 was from R&D Systems. Cisplatin (Sigma-Aldrich) was diluted in sterile PBS and used to a final concentration of 25 μmol/L.

Exosome Purification:

B16F10, CT26, HCT116 and SW480 cells were cultured for 48 h or 24 h with cisplatin (CDDP) treatment in medium depleted from serum-derived exosomes by overnight centrifugation at 100,000 g. Then, supernatants were collected from cell lines and sequentially centrifuged at 300 g for 10 minutes (4° C.) and then at 2000 g for 10 minutes. Exosomes were isolated at 100,000 g for 70 minutes and washed once in PBS. Pellets were resuspended in 200 μl of PBS. The concentration of exosomal proteins was quantified by Lowry assay (Biorad).

Human urine and mice blood samples were collected and sequentially centrifuged at 2,000 g for 30 min (4° C.) and then at 12,000 g (4° C.). Exosomes were isolated at 100,000 g for 70 minutes and washed once in PBS. Pellets were resuspended in 100 μl of PBS.

Immunoprecipitation:

For the in vitro coimmunoprecipitation, we used A8 or TLR2 recombinants proteins. HSP70WT and HSP70ΔABD (both HA-tagged) were produced with the TNT Quick Coupled Transcription/Translation System (Promega) as follows: 1 mg of template plasmid was added to the reaction mixture and incubated at 30° C. for 90 minutes. TLR2 was incubated with HSP70 alone or together with A8 (from 0.3 μM to 1.8 μM). The immunoprecipitated proteins were analysed by western blot.

Western Blotting:

Proteins were separated in an 8% or 10% SDS-polyacrylamide gel and transferred to nitrocellulose membranes using a wet transfer apparatus (Bio-Rad). Membrane were saturated 1 h with 5% (w/v) non-fat dry milk or BSA, and probed overnight with the corresponding primary antibodies: anti-HA-tag was from Covance (Eurogentec), anti-thioredoxin (Sigma), anti-TLR2 (Santa Cruz), anti-HSP70 (Enzo Life), anti-Stat3 (Santa Cruz), anti-pSTAT3 (Cell signalling), anti-CD81 (Ebioscience), anti-flotilin-1 (BD Biosciences) and anti-14-3-3 (Santa Cruz). Then, the membranes were incubated for 1 h with appropriate secondary antibodies coupled to horseradish peroxidase (Jackson ImmunoResearch Laboratories) and revealed with ECL (Amersham).

Transmission Electron Microscopic (TEM):

The exosomes suspension (30 μl) was added to a collodion-carbonated nickel-200 grids and fixed with paraformaldehyde 4%. The sample was then saturated 10 minutes with PBS NGS2%, washed and labelled with cmHSP70 (Multimmune, Germany) during to 2 hours. After washing with PBS supplemented with 1% BSA, exosomes were labelled with a secondary antibody coupled with a 10 nm gold beads. A post-fixation step was performed with a 2% glutaraldehyde solution and then stained with 3% phosphotungstic acid. The sample was analysed with a Hitachi H-7500 electronic microscope equipped with an AMT camera.

Flow Cytometry:

Exosomes were bound to surfactant-free white aldehyde-sulfate latex beads (3 μM, Invitrogen) and concentrated at 1·10⁴ beads/pt. Exosomes (30 μg) were incubated with the beads suspension for 15 min and then for 1 h at room temperature (RT) in final volume of 1 mL PBS. Beads were saturated with glycine (100 mM) for 30 minutes at RT and washed twice with PBS supplemented by 3% of FBS. Exosomes or cancer cells were labelled by a classical staining with control isotype or cmHSP70-FITC (Multimmune, Germany) antibodies. Analyses were performed on a LSRII flow cytometer (BD, Bioscience).

Biolayer Interferometry (BLI):

Protein-protein interaction experiments were conducted at 25° C. in PBS with an Octet Red instrument (Forte Bio, Menlo Park, Calif., USA) by biolayer interferometry (BLI). The ligand (cmHSP70 or A8 or TLR2) was biotinylated using EZ-Link NHS-PEG₄-biotin (Thermo Fisher Scientific, Bonn, Germany) according to the manufacturer's protocol and immobilized at a concentration of 20 μg/ml on streptavidin sensors (ForteBio, Menlo Park, Calif., USA). These functionalized biosensors were dipped into black Greiner 96-well microplates agitated at 300 g and filled with a solution of 200 μL containing the analyte. All sensorgrams were corrected for baseline drift by subtracting a control sensor exposed to running buffer only. Each K_{D} was determined with a 1:1 stoichiometry model using a global fit with Rmax unlinked by sensor (Forte Bio, Data analysis software version 7.1.0.89).

MSC2 Functional Assay:

Five millions of MSC2 cells were incubated with or without exosomes isolated from B16F10, CT26, HCT116 or SW480 cells in the presence or absence of A8 during 4 h or 6 h. Cells were isolated, lysed and analysed for pSTAT3 by western blotting.

Tumor Growth Analysis In Vivo:

Exponentially growing B16F10 were harvested and resuspended in an RPMI medium without FBS. In vivo studies were performed in wild-type C57/BL6 mice (Charles River). B16F10 cells (5·10⁴) were injected s.c. into the right flank Tumor volumes were evaluated every 2 days. Mice were treated with a control peptide aptamer A0 (3 mg/kg) or the HSP70 aptamer A8 (3 mg/kg) and half mice were treated with cisplatin (CDDP, 5 mg/mL). The animals were treated according to the guidelines of the Ministère de la Recherche et de la Technologie, France. All experiments were approved by the Comité d'Ethique de l'Université de Bourgogne.

MDSC cell isolation and analysis: Single-cell suspensions were prepared from spleens, and red cells were removed using ammonium chloride lysis buffer. Gr-1+ cells were isolated from spleens of different group of tumor-bearing mice or naive mice by labeling the cells with PE Cy7 Ab to Gr-1, then using magnetic PE Cy7 beads and LS MACS columns (Miltenyi Biotec). For extracellular staining of immune markers, single-cell suspensions were prepared. We incubated 1·10⁶ freshly prepared cells with fluorochrome-coupled CD11b antibody (eBioscience). All events were acquired by a BD Bioscience LSR-II device and analyzed with FlowJo (Tree Star).

Immunofluorescence:

B16F10 (25·10³ cells), HCT116 (5·10⁴ cells) and MEF (3·10⁴ cells) were seeded in 24 well-dished which was pretreated for 10 minutes with Poly-L-Lysin (P4707, Sigma Aldrich). 24 h later, cells were washed with PBS and labelled with cmHSP70 antibody (1:200) for 1 h at 4° C. After washing, cells were fixed with 4% paraformaldehyde at 4° C. for 10 min and washed once. Then, cells were saturated for 30 minutes with BSA 3% and incubated with a secondary antibody labelled with Alexa-568 (1:1000) at 4° C. for 1 h. Cover glasses were mounted on a drop of Mounting Medium containing Dapi (Duo82040, Sigma Aldrich) for 15 minutes in the dark on a microscopy slide (045796, Dutscher). Slides were imaged using a CDD equipped up right microscope (Zeiss) and 63×, 1.4NA objective.

Histologic Study of the Tumor:

Animals were killed 18 days after cell injection. The site of tumor cell injection was resected and snap-frozen in liquid nitrogen. An immunohistochemical study of tumor-infiltrating inflammatory cells was performed on acetone-fixed 5 μm cryostat sections.

ELISA (Enzyme Linked Immunosorbent Assay):

MSC2 cells were incubated alone or co-incubated with exosomes from B16F10 cells and/or A8 (during 24 h). Then, the supernatant was collected and centrifuged at 300 g at 4° C. for 5 min and analyzed with IL-6 ELISA assay (BD biosciences) according to the manufacturers' protocol.

Analysis of Exosomes Concentration:

After ultracentrifugation, suspensions containing exosomes were analysed using a NanoSight LM10 instrument (Nanosight, Amesbury, UK). For this analysis the samples were dilued in PBS. Three videos of 60 s duration were taken with a frame rate of 30 frames/s and with a syringe pump at 70 μl/min at 20° C., and particule mouvement under Brownian motion was analysed by NTA software (version 2.3 NanoSight).

Refolding Luciferase Assay:

Denatured luciferase was incubated in the presence or absence of human HSP70/HSP40 and/or A8 (from 5 μM to 50 μM) for 1 hour at 25° C. at the following ratios (luciferase:HSP70:HSP40 (1:10:2). Following incubation, the luciferase substrate D-luciferin was added and the total light units emitted were collected for 10 seconds at 560 nm using a Wallac (Victor 3) spectrophotometer. (n=3).*, p<0.05.

HSP70 ELISA Assay:

Exosomes were lysed with Ripa buffer. Then, the lysate were analyzed with home-made ELISA assay.

Statistical Methods:

Results are expressed as means±SEM from at least three independent experiments. Values were analyzed using student's t test. All p values were obtained using two-tailed tests and error bars in the graphs represent 95% confidence intervals. Quantitative data were analyzed using the GraphPad Prism program. Statistical significance, p<0.05, are denoted with *.

Results

Quantification of HSP70-Exosomes in Cancer Patients' Samples Using A8 Peptide Aptamer as a Ligand:

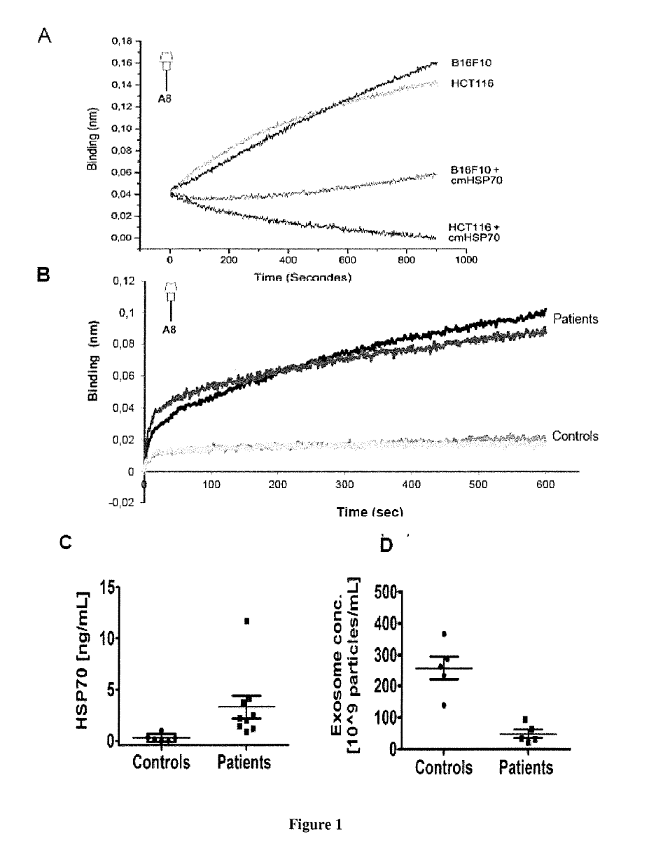

In Chalmin et al, we previously showed that exosomes from CT26 mouse colon cancer cell line expressed HSP70 on their membrane (HSP70-exosomes) which was responsible to suppress anti-cancer immune response by MDSC activation [12]. When HSP70 anchors in the plasma membrane only a TKD sequence in the peptide binding domain is extracellular [34]. In non-permeated cells, membrane HSP70 can therefore only be detected with antibodies specifically targeting this TKD region such as the commercial monoclonal cmHSP70. We previously described the selection intracellular inhibitors of HSP70, called peptide aptamers [26]. One of them, a 8 amino-acid peptide aptamer we called A8, binds within the C-terminal region of HSP70 [26]. We wondered whether A8 could precisely associate to the extracellular TKD sequence of membrane-bound HSP70 and therefore could be used to capture tumor-derived exosomes. To answer this question, we analyzed its ability to bind to extracellular HSP70 in the membrane of tumor-derived exosomes. We demonstrate by biolayer interferometry (BLI) that A8 efficiently captured exosomes though its interaction with membrane-HSP70 and that, in a dose dependent manner, decreased the ability of cmHSP70 to bind to HSP70, indicating that both molecules may compete for membrane HSP70 binding (FIG. 1A). The advantage of A8 over the cmHSP70 antibody, other than its potential therapeutic use, is that peptide aptamers are smaller in size (an important advantage for detection using optical devices), stable, soluble, easy to produce and elicit little immune response.

We next set up a biolayer interferometry (BLI) protocol where we used A8 as a ligand to determine whether we could capture HSP70-exosmes from human samples. We analyzed cancer patients (metastatic breast, lung and ovary cancers; n=9) and healthy donors (n=5) and for practical reasons we used urine samples. The results found with this precise and fast protocol (FIG. 1B) were confirmed by customized ELISA analysis (FIG. 1C). With both techniques we found similar results: the number of HSP70-exosomes was significantly higher in all cancer patients compared with the healthy donors where hardly no HSP70-exosomes could be detected (FIGS. 1B and 1C). These interesting results were highlighted by the fact that the total number of exosomes quantified by NanoSight found in the urines was in fact higher in healthy donors than cancer patients (FIG. 1D), showing the particular interest of quantifying specifically this sub-set of nanovesicles that are the HSP70-exosomes.

HSP70-Exosomes are Tumor-Derived Exosomes:

To study how specific markers are HSP70-exosomes of tumor-derived exosomes we determined in cultured cells if HSP70 presence in the membrane of tumor-derived exosomes was a general feature of cancer cells.

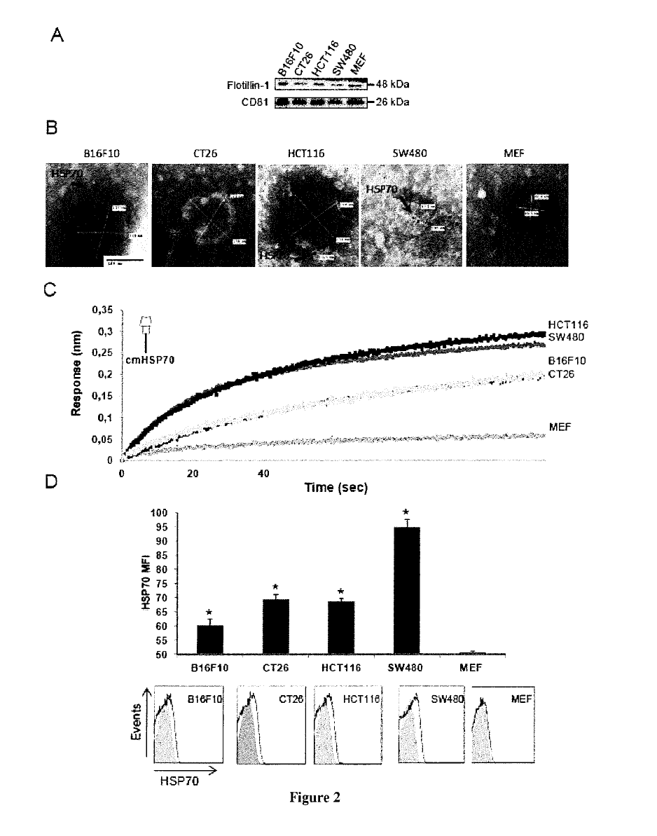

Diverse techniques were used to detect the presence of HSP70 at the surface of exosomes in different human and rodent cancer cell lines. We evaluated the release of exosomes in culture media from 4 cancer cell lines (HCT116 and SW480 human colon cancer, CT26 mouse colon carcinoma and B16F10 mouse malignant melanoma) and in two non-cancerous cell lines (MEF: mouse embryonic fibroblast and PrCE: human prostate cells). First, to avoid any artifact related to the presence of others micro-vesicles potentially co-purified with exosomes, we validated the expression of specifics classical markers of exosomes in all samples such as Flotillin-1 and CD81 (FIG. 2A). Next, we used a standard transmission electronic microscope (TEM) to characterized TDE. As shown in FIG. 2B, we were able to detect secreted exosomes in culture media from all cells, both cancerous and non-cancerous. However, extracellular HSP70 was detectable at the surface of exosomes only in those derived from cancer cell lines (B16F10, CT26, HCT116 and SW480, FIG. 2B). This result was confirmed by two other techniques i.e biolayer interferometry (BLI, OctetRED instrument, FortéBio) (FIG. 2C) and flow cytometry (FACS) (FIG. 2D).

Paralleling the analysis of membrane-bound HSP70 in the exosomes, we checked the expression of HSP70 in the plasma membrane of the different cells by immuno fluorescence and FACS analysis. We observed that, as already reported [31], membrane-bound HSP70 was only detected in cancer cell lines but not in normal cells. This could explain why release of exosomes presenting HSP70 at their membrane may be a general feature of a cancer cell.

A8 Peptide Aptamer Blocks HSP70 Ability to Associate to TLR2:

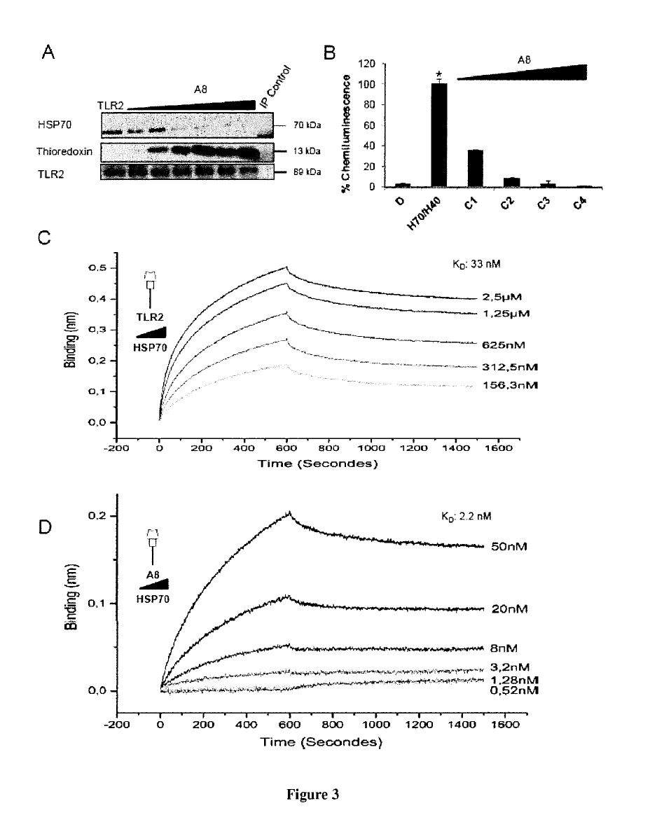

Activation of MDSC by exosomes is mediated via the binding of extracellular HSP70 to the TLR2 expressed on MDSC [12]. We therefore studied whether A8 could interfere with HSP70/TLR2 interaction. As shown in FIG. 3A, we found that HSP70 association to TLR2 was inhibited by A8 in a dose-dependent manner (from 0.3 μM to 1.8 μM). A8 also blocked, in a dose-dependent manner, HSP70 chaperone activity as measured by a luciferase refolding assay using purified recombinant proteins (FIG. 3B).

To further characterize the effect of A8, we measured constants of dissociation (K_{D}) by BLI. We immobilized A8 or TLR2 on the surface of the biosensor and determined their K_{D} for HSP70. The K_{D} of HSP70 for TLR2 and A8 was 33 nM and 2.2 nM, respectively (FIGS. 3C and 3D). These data indicate that HSP70 displays a lower affinity for TLR2 than for A8, which could explain why A8 efficiently prevents the interaction between HSP70 and TLR2.

A8 Blocks the Ability of Tumor-Derived Exosomes to Activate MDSC:

The TLR2-dependent activation of MDSC by exosomes involves the production and release of IL-6 [35]. Once released, IL-6 triggers the activation of its receptor and the phosphorylation of STAT3 [36]. Therefore, we evaluated the ability of A8 to prevent the activation of MDSC by exosomes by determining IL-6 secretion and STAT3 phosphorylation status.

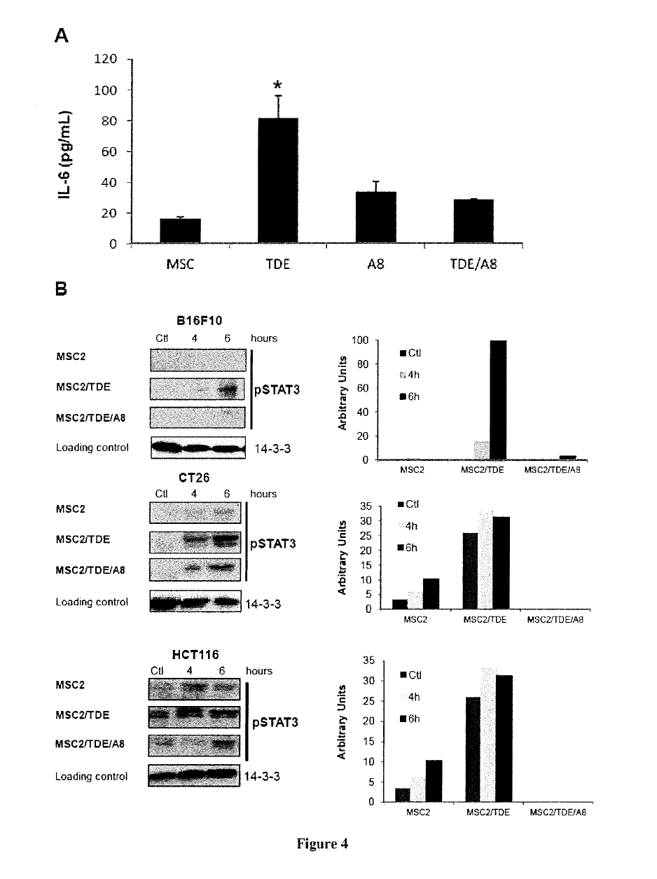

MSC2 (myeloid suppressive cells 2) were incubated in the presence or absence of tumor-derived exosomes (isolated from B16F10 cells) and/or A8. Supernatants were harvested and secreted IL-6 was quantified by ELISA. We found that, as expected, exosomes derived from tumor cells stimulate the secretion of IL-6 induced by MSC2 cells. Importantly, this IL-6 production was abrogated by A8 (FIG. 4A).

Then, we checked the activation of the JAK2/STAT3 signaling pathway in these MDSC cells, by analyzing STAT3 phosphorylation by western-blot. MSC2 cells were incubated in the presence of exosomes isolated from the supernatant of the four different cancer cell lines (CT26, B16F10, HCT116 and SW480) with or without A8. In the absence of tumor cells-derived exosomes, we did not observe any phosphorylation of STAT3 in MSC2, indicating that they are not activated at basal level and that A8 does not have a direct effect in MDSC activation. In contrast, the presence of tumor-derived exosomes (TDE) from any of the four cancer cells analyzed induced STAT3 phosphorylation at 4 h and 6 h of incubation (FIG. 4B). Addition of A8 in the culture medium inhibited the ability of tumor-derived exosomes to activate STAT3 in MSC2. This effect was reproducible since a similar inhibitory effect was obtained with exosomes isolated from the 4 cancer cell lines analyzed in this work (FIG. 4B).

To further prove that the inhibitory effect of A8 on MSC2 activation was due to its direct interaction with the extracellular C-terminal region of HSP70 at the exosomes surface, we tested the effect of another peptide aptamer called A17. We demonstrated in the past that this peptide aptamer binds to the ATP binding domain of HSP70, which is located in the intracellular N-terminal region of membrane-bound HSP70 [26]. The peptide A17, in contrast to A8, did not interfere with the ability of tumor-derived exosomes to induce the activation of STAT3 in MCS2.

We also checked by flow cytometry whether A8, when incubated with tumor-derived exosomes could affect the survival of MSC2. A8 alone or together with tumor-derived exosomes, did not induce any significant cell death in MSC2.

Thus, these results demonstrate that A8, by its interaction with the extracellular domain of HSP70 in the tumor-derived exosomes from human and rodent cell lines, blocks their ability to induce the activation of MDSC through the TLR2 signaling pathway.

A8 Prevents the Activation of MDSC In Vivo and Improves Cisplatin Chemotherapeutic Effect:

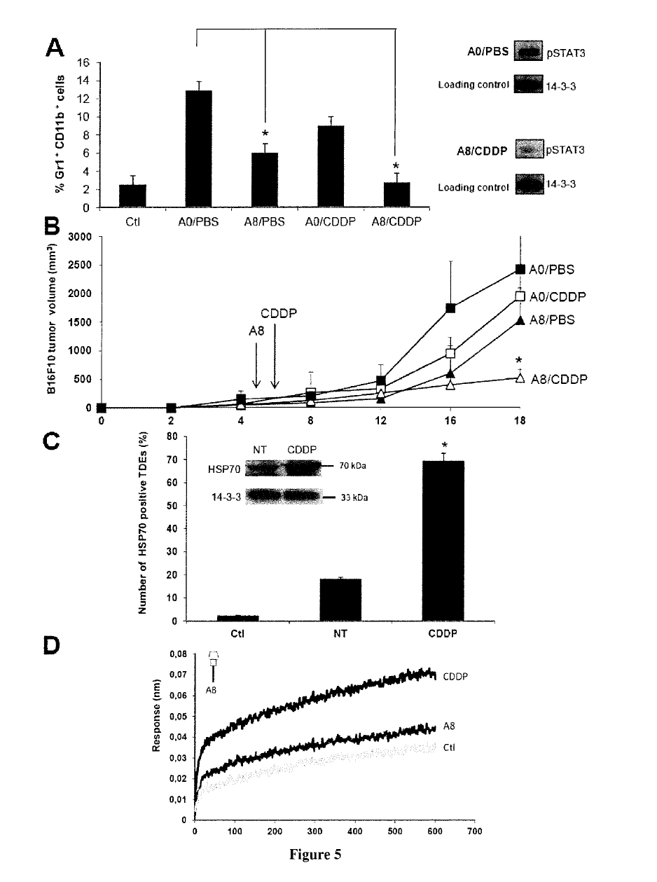

Next, we tested whether A8 was also able to block the immunosuppressive effect of MDSC in an in vivo cancer model. We performed subcutaneous injection at day 0 of B16F10 cells or media as a control in C57/BL6 mice. When tumor size reached about 0.9 mm³ (by day 5), mice were treated i.p. with a control peptide aptamer (A0) or A8 every two days until the end of the experiment. When indicated, half of the animals received cisplatin (CDDP, 10 mg/Kg, i.p.) as a single dose. Tumor growth was measured every two days. After 18 days, we analyzed the percentage of MDSC present in mice spleen (6 animals per group) by FACS using the specific markers Gr1⁺ and CD11b⁺ (FIG. 5A). In agreement with the literature [37], we found 2.5% of MDSC in the spleen of mice with no tumors (control group: Ctl), whereas in mice bearing a tumor and treated with A0 we observed a five-fold increase in the percentage of MDSC (12.9+/−1.2). Importantly, this increase was strongly decreased in mice bearing tumors that were treated by A8 (6+/−0.7, FIG. 5A). In animals treated with cisplatin alone, the percentage of MDSC was 9+/−0.9, whereas in the spleen of tumor-bearing mice treated with both A8 peptide and cisplatin only barely 2.7+/−0.8% (n=6) of MDSC were found. Remarkably, this percentage of MDSC reached after A8 and cisplatin treatment in the spleen of the animals bearing a tumor was similar to that found in animals bearing no tumors (Ctl, FIG. 5A). A8 not only induced a decreased the number MDSC after cisplatin treatment but also, as expected, affected their activity as determined by measuring phosphorylated STAT3 (FIG. 5A). Indeed, the effect of A8 in the number of MDSC is most probably a consequence of its above-mentioned effect blocking their activation and thereby the IL-6-dependent proliferation loop [38].

A8 blocking effect on MDSC was associated with a strong increase in the anti-cancer properties of cisplatin (e.g. in the representative experiment shown in FIG. 5B. 5 out of the 6 animals tested were tumor-free when cisplatin was associated to A8). The decrease in MDSC induced by A8 not only correlated with tumor regression as shown in FIG. 5B, but also with an intra-tumor infiltration of immune cells notably T cells (CD3⁺), dendritic cells (CD11c⁺), monocytes (CD11b⁺) and macrophages (F4/80⁺).

Since, as we showed previously, A8 blocked the capacity of HSP70-exosomes to activate MDSC, we next determined whether cisplatin increased the amount of HSP70-exosomes, which could explain why its combination with A8 strongly increased cisplatin anti-cancer properties. We isolated total exosomes both from cultured B16F10 cells and from the blood of mice bearing a tumor by ultracentrifugation. Then, we quantified HSP70-exosomes respectively by FACS (FIG. 5C) and by using our BLI protocol in which A8 serves as a ligand (FIG. 5D). We found that the amount of HSP70-exosomes released by B16F10 cancer cells strongly increased after cisplatin treatment and, confirming these results, the amount of HSP70-exosmes in the blood was much stronger in the CDDP-treated animals (n=3, FIG. 5D). These results suggest that quantifying HSP70-exosomes in different therapeutic set ups could help determining the chemotherapeutic treatment which, like cisplatin, may benefit the most from our proposed HSP70 peptide aptamer approach.

Altogether, our in vitro and in vivo results show that A8 peptide can directly block the effect of HSP70-exosomes activating MDSC by inhibiting HSP70/TLR2 interaction and thereby the STAT3 pathway. As a result, immunosuppressive MDSC can be impaired and the anti-tumor immune response might be restored. We also set up an A8-BLI protocol to demonstrate that HSP70-exosomes can be easily quantified both from urines and blood samples and might be an interesting tool to quantify tumor-derived exosomes.

Discussion

HSP70 expressed in the membrane of exosomes is involved in MDSC activation [12]. MDSC have been subject of a particular scrutiny for playing a critical role in cancer progression [39]. Here, we show by analyzing different rodent and human cells that the presence of HSP70 in the membrane is probably a general character of exosomes released from tumor cells. Cancer cells accumulate mutations, violate physiology laws and acquire sets of hallmarks and therefore require a constitutively high level of chaperones like HSP70 for their survival/maintenance [40]. Since only 10% of the total amount of intracellular HSP70 is expressed at the cytoplasmic membrane, this most likely explains why exosomes from normal cell are devoid of membrane-bound HSP70. Yet, how this membrane anchorage takes place is still unknown. However, it has been demonstrated that a 14 amino-acid region (called TKD domain) of the C-terminus domain of HSP70 remains extracellular [29].

There is a growing body of evidences indicating that an efficient chemotherapy must rely on an efficient anticancer immune response [41,42]. In this way, cisplatin has been described as a non-immunogenic drug with a limited anti-tumor efficacy [43]. A partial explanation given in the past for the inability of cisplatin to induce an efficient anti-cancer immune response was its incapacity to favor the translocation of immunogenic calreticulin onto the plasma membrane of the cancer cell [44]. In this work, we propose another explanation involving HSP70 and MDSC. We show that cisplatin treatment—already described to induce HSP70 expression in cancer cells [45]—increases the amount of exosomes expressing HSP70 on their surface and, probably as a consequence, the amount/activity of MDSC. Whether HSP70 and calreticulin translocation into the plasma membrane are two connected events remains to be explored.

With the aim of blocking HSP70 on the exosomes and thereby their ability to activate MDSC, we have selected a peptide aptamer, A8, that binds to an extracellular domain of membrane-bound HSP70. A8 binds with high affinity to the extracellular domain of HSP70 on the exosomes, blocks HSP70 association to MDSC receptor TLR2 in a dose dependent manner and, thereby, MDSC activation. By doing so and thereby hampering IL-6 production, A8 also blocked MDSC proliferation [38], explaining the reduction in MDSC found in the animals bearing tumors that was particularly strong after cisplatin treatment. Thus, this peptide inhibitor of HSP70 by interfering with the immune suppressive functions of tumor-derived exosomes may thus improve the efficacy of an anticancer drug such as cisplatin. Indeed, we demonstrated that cisplatin combined with A8 decreased tumor growth and favored the development of an anticancer immune response. The advantages of peptides aptamers over a HSP70 antibody targeting also an extracellular domain of HSP70 (e.g. TKD) like cmHSP70, besides the tolerances issues antibodies can induce, are that peptides are much smaller and easier to produce [33]. Interestingly, A8 biodistribution experiments (SPWPRPTY peptide of SEQ ID NO: 2), after systemic administration, demonstrate that the peptide localizes mainly in the tumor area [46], which is likely due to the fact that tumors abundantly express HSP70. Therefore, a peptide like A8 confers greater tumor specificity than for instance folic acid. Considering the general role of HSP70 in different cancers and its induction by a large panel of anti-cancer agents, the anti-cancer immune approach proposed in this work involving inhibition of HSP70-exosomes might be extended to different cancer types with their chemotherapeutical set-ups. It is worth noting that neither our studies nor those done for the bio-distribution of A8 indicate any evident toxicity in the animals, notably cardiac or hepatic.

Finally, we set up a protocol to demonstrate that HSP70-expressing exosomes could be captured from cancer patients' urines samples using as ligands either the antibody cmHSP70 or peptide aptamer A8, that both recognise the extracellular domain of membrane-bound HSP70. Other than the rational for a combinational therapy, our results in human samples suggest that HSP70-exosomes could be used as a biomarker for cancer. Nowadays the detection of tumour circulating cells (CTCs) is used for diagnosis purposes during patients' follow up. The approach has three important drawbacks: 1) the presence of CTCs in the blood is a rare event (only about 1/10⁹ cells is a CTCs) 2) the detection method (CellSearch) is based on the acquisition of many images (hundreds) whose analysis has to be done by an expert (subjectivity). 3) The marker used, EpCAM is not present in all CTCs (for instance those from melanomas). These three problems can be solved if HSP70-exosomes (cancer-derived exosomes) are measured instead of CTCs (or together with CTCs). First, several hundreds of exosomes can be released by a single cancer cell and, therefore, we have an amplification cancer signal. Second, the detection method we propose is faster and more objective than any image analysis (e.g. Cell Search analysis). Finally, we proposed HSP70 as a universal marker of tumour cells to perform.

It is possible that the cancer-associated abundant expression of HSP70 occurs quite early during the tumorigenic cell transformation process and, as explained in this work, could be a protection strategy developed by the malignant cell to escape the immune surveillance.

A prospective study has been started with the anticancer Centre Georges-Francois Leclerc (CGFL, Dijon, France) in breast and lung cancer patients to determine whether the presence of exosomes presenting HSP70 in the membrane is predictive of the patients' outcome and whether its detection precedes that of CTCs (CellSearch) and the apparition of metastases (scanner).

### Example 2

Material & Methods

Cells and Products:

B16F10, CT26, HCT116, SW480, PC3, HeLa, EL4 cancer cell lines (American Type Culture Collection, ATCC) were cultured in RPMI 10% foetal bovine serum (FBS, Lonza), Mouse embryonic fibroblasts (MEF) were cultured in DMEM 10% FBS (Lonza). All human primary cells: Prostate Epithelial Cells (PrEC, ATCC), Primary Uterine Cells (PUC, from biopsies of myometrial cells, CHU Dijon), Normal Colon Mucosa (NCM, INCELL corporation LLC) and Mammary Epithelial Cells (HMEC), were cultured in specific cell basal medium. MSC2 cell line (myeloid suppressor cell 2), a gift from V. Bronte (Instituto Oncologico, Padova, Italy), was cultured in RPMI 10% FBS. All cells were cultured with 5% of CO₂ at 37° C.

The peptide aptamer A8 (SPWPRPTY) and A17 (YCAYYSPRHKTTF) were synthesized and highly purified by Proteogenix (France). Monoclonal antibody cmHSP70 was purchased from Multimmune, Germany and recombinant HSPs from StressGen (EnzoLife), TLR2 from R&D Systems and cisplatin, 5FU, paclitaxel, doxorubicin, gemcitabine from Sigma-Aldrich.

Exosome Purification:

Cells were cultured in medium depleted from serum-derived exosomes. Supernatants were collected from cell lines and sequentially centrifuged at 300 g for 10 minutes (4° C.) and at 2,000 g for 10 minutes. Then, exosomes were ultracentrifugated at 100,000 g for 70 minutes and washed in PBS. The same protocol was used for urines and blood samples. To evaluate exosome concentrations, we used a Nano Sight LM10 instrument (NanoSight, Amesbury, UK).

Biolayer Interferometry (BLI):

Protein-protein interaction experiments were conducted with an Octet Red instrument (ForteBio, USA). The ligand (A8 or TLR2) was biotinylated using EZ-Link NHS-PEG₄-biotin (2 nM, 30 min, RT, Thermo Fisher Scientific, Germany) and immobilized on streptavidin sensors (96-well plate black, ForteBio, USA). Functionalized sensors were incubated in PBS (10 min) then incubated with exosomes (10⁶, from cancer cells lines or normal cells or mice blood or human urine, for 120 or 600 seconds) or HSP70 (concentration range from 2.5 μM to 0.52 nM) or cmHSP70 (1/1,000). All sensorgrams were corrected for baseline drift by subtracting a control sensor exposed to running buffer only. Each K_{D} was determined with a 1:1 stoichiometry model using a global fit with Rmax unlinked by sensor (ForteBio, Data analysis software version 7.1.0.89).

Western Blotting:

Proteins were separated in an 8-10% SDS-polyacrylamide gel and transferred to nitrocellulose membranes as already described²³. Primary antibodies used: HA-tag (1/2,000, monoclonal, Covance, Eurogentec), thioredoxin (1/2,000; polyclonal, Sigma), TLR2 (1/1,000; Santa Cruz), HSP70 (1/2,000; polyclonal Enzo Life), STAT3 (1/1,000; polyclonal, Santa Cruz), pSTAT3 (1/1,000; monoclonal, Cell signalling), CD81 (1/1,000; polyclonal Abgent), flotillin-1 (1/1,000; monoclonal, BD Biosciences) and 14-3-3 (1/1,000; polyclonal, Santa Cruz). After incubation with appropriate secondary antibodies (1/20,000) coupled to horseradish peroxidase (Jackson ImmunoResearch Laboratories) membranes were revealed with ECL (Amersham).

Immunoprecipitation:

For the in vitro co-immunoprecipitation, we used A8 or TLR2 recombinants proteins. HSP70WT and HSP70ΔABD (both HA-tagged) were produced with the TNT Quick Coupled Transcription/Translation System (Promega) as follows: 1 mg of template plasmid was added to the reaction mixture and incubated at 30° C. for 90 minutes. TLR2 was incubated with HSP70 alone or together with A8 (from 0.3 to 1.8 μM) overnight at 4° C. The immunoprecipitated proteins were analyzed by western blot.

Transmission Electron Microscopic (TEM):

The exosomes suspension (30 μl) was added to collodion-carbonated nickel-200 grids and fixed with paraformaldehyde 4%. The sample was saturated with PBS NGS2%, washed and labelled (2 h) with cmHSP70 (1/50, monoclonal, Multimmune, Germany). After washing with PBS supplemented with 1% BSA, exosomes were labelled with a secondary antibody coupled with 10 nm gold beads. A post-fixation step was performed with a 2% glutaraldehyde solution and then stained with 3% phosphotungstic acid. The sample was analyzed with a Hitachi H-7500 electronic microscope equipped with an AMT camera.

Flow Cytometry:

Exosomes from cancer cells lines or normal cells were bound to surfactant-free white aldehyde-sulfate latex beads (3 μM, Invitrogen) and concentrated at 1·10⁴ beads/μL. Exosomes (30 μg) were incubated with the beads suspension for 15 min and then for 1 h at room temperature (RT) in final volume of 1 mL PBS. Beads were saturated with glycine (100 mM) for 30 minutes at RT and washed twice with PBS supplemented by 3% of FBS. Expression of membranar HSP70 on cells (normal and cancerous) was determined by flow cytometry using cmHSP70 antibody. MSC2 and HCT116 cell death was measured by PS exposure, cells stained with 7AAD and FITC-Annexin V conjugate. All analyses were performed on a LSRII flow cytometer (BD Bioscience).

pSTAT3 Analysis:

MSC2 (2·10⁶) or MDSC (2·10⁶) were incubated alone or with exosomes (from B16F10, CT26, HCT116, SW480) in the absence or presence of A8 or A17 (16 μM) during 6 hours (RPMI 10% FBS, 5% of CO₂ at 37° C.). Every 2 hours, 5·10⁶ cells were sampled and centrifugated 5 min at 300 g. Cells were lysed in lysis buffer (50 mM HEPES (pH 7.6), 150 mM NaCl, 5 mM EDTA, and 0.1% NP40 and anti-phosphatases, Roche). Then, pSTAT3 and STAT3 expression were analyzed by western blotting.

MDSC Cell Isolation and Analysis:

Single-cell suspensions were prepared from spleens, and red cells were removed using ammonium chloride lysis buffer. Gr-1+ cells were isolated from spleens of different group of tumor-bearing mice or naive mice by labeling the cells with PE-Cy7 Ab to Gr-1, then using magnetic PE-Cy7 beads and LS MACS columns (Miltenyi Biotec). For extracellular staining of immune markers, single-cell suspensions were prepared. We incubated 1·10⁶ freshly prepared cells with fluorochrome-coupled CD11b antibody (eBioscience). All events were acquired by a BD Bioscience LSR-II device and analyzed with FlowJo (Tree Star).

Lymphocytes Isolation and Co-Culture Experiment:

Single-cell suspensions were prepared from spleens, and red cells were removed using ammonium chloride lysis buffer. Lymphocytes were isolated from spleens of naive mice in a two step procedure. First, non-CD4+ T cells are indirectly magnetically labeled. The labeled cells are subsequently depleted by separation over a LS Column (Miltenyi Biotec). In the second step, T cells using magnetic CD62L beads and LS MACS columns (Miltenyi Biotec). Then, purified lymphocytes (1.10 were activated with CD3/CD28 beads (Fisher) and rapidly incubated with primary MDSC (1·10⁴) (see MDSC cell isolation section) alone or with exosomes (from B16F10) in the absence or presence of A8 (16 μM). At day 5, supernatants were collected, centrifuged (300 g, 5 min) and analyzed by ELISA (see ELISA section).

ELISA (Enzyme Linked Immunosorbent Assay):

Supernatants, collected from MSC2 or MDSC or co-cultured cells incubated alone or with exosomes from B16F10 cells and/or A8 (8 or 16 μM) were analyzed with IL-6, IL-10 and IFN gamma ELISA assay (BD biosciences) according to the manufacturers' protocol.

Refolding Luciferase Assay:

Denatured luciferase was incubated in the presence or absence of human HSP70/HSP40 and/or A8 (from 5 μM to 50 μM) for 1 hour at 25° C. at the following ratios (luciferase:HSP70:HSP40 (1:10:2). Following incubation, the luciferase substrate D-luciferin was added and the total light units emitted were collected for 10 seconds at 560 nm using a Wallac (Victor 3) spectrophotometer.

Histologic Study of the Tumor:

Animals were killed 15 or 18 days after cell injection. The site of tumor cell injection was resected and snap-frozen in liquid nitrogen. An immunohistochemical study of tumor-infiltrating inflammatory cells was performed on acetone-fixed 5 μm cryostat sections. Tumoral biopsies were blocked in PBS with 3% of BSA (10 min, RT) anti-CD11c (1/50, monoclonal, BD Pharmingen), anti-CD3 (1/50, monoclonal, BD Pharmingen), anti-F4/80 (1/100, monoclonal, BD Pharmingen), diluted in PBS with 1% of BSA, was added on slides and incubated 45 minutes at RT, after washing with PBS. Nuclei were colored with mounting medium: Prolong Gold with Dapi (Molecular Probe). The slides were observed with Axio Imager 2 (Carl Zeiss Microscopy GmbH, Jena, Germany) equipped with an Apotome.2 module (Carl Zeiss GmbH). Images were acquired using an AxioCam MRm monochrome CCD camera (Carl Zeiss GmbH). Six pictures were taken in random fields for each labeling, and analyzed using image J software.

Concerning macrophage characterization (M1-like), DHE (Dihydroethidium, 10 μM, Thermo Fisher) was added on slides after fixation, and incubated 10 minutes at RT. For immunofluorescence analysis, samples were blocked/permeabilized in PBS, 1% BSA and 0.1% saponin (10 min, RT) and incubated overnight at 4° C. with the primary anti-p-STAT1 antibody (1/200; polyclonal, Cell Signaling), or anti-SOCS1 (1/200, polyclonal, Santa Cruz), or anti-NOS2 (1/200, polyclonal Santa Cruz). Primary antibodies were detected using Anti-FITC (1/300) or Alexa fluor488 or Alexa fluor647-coupled secondary antibodies (1/500, Invitrogen), 30 minutes at RT. Nuclear labeling was performed by incubating samples in DAPI (1 ng/mL). The slides were observed with a Nikon (E400, Eclipse) epifluorescence microscope.

MSC2 and MDSC Cell Proliferation Analysis:

MSC2 cells proliferation were analyzed by to different techniques. First, MSC2 (1·10⁴) were seeded onto 16-well plates with medium containing 10% FBS and incubated for 34 hours. Then, cells were treated or not with exosomes (from B16F10) in the absence or presence of A8 (16 μM) during 5 days. Into each well of the micro-electronic sensor transendothelial electric resistance (TER) was measured by xCELLigence real time cell analyzer DP system (Roche). TER was measured every 30 s and then normalized to the initial value. Secondly, MSC2 (7·10⁵) cells were treated or not with exosomes (from B16F10) in the absence or presence of A8 (16 μM) during 3 days. Then, cells were fixed and permeabilized (Cytofix/Cytoperm, BD Biosciences) and stained by BrdU (20 min, RT). After a washing step, cells were resuspended into staining of DNA for cell cycle analysis using 7AAD and analyzing on a flow cytometer LSRII (BD Biosciences). MDSC proliferation was analyzed by cell counting. MDSC cells (1·10⁶) from spleen of naive mice (see MDSC isolation section) were treated or not with exosomes (from B16F10) in the absence or presence of A8 (16 μM) for 24 hours and then analyzed for viability by Cellometer-mini (Ozyme).

Surface Plasmon Resonance:

Measurements of A8:HSP70 interactions were performed by surface plasmon resonance (BIAcore BX100, General Electrics) at 25° C. with a standard CM5 chip. HSP70 was covalently coupled to a CM5 chip surface with primary amine groups using standard amine coupling (EDC/NHS chemistry method). A8 solutions (12.8 or 25.6 μg/mL) (n=3) were prepared in HBS EP+ and injected successively from the lower to the higher concentration.

Results:

HSP70-Exosomes are Universal Markers of Cancer Cells and of Response to the Chemotherapy.

To prove the universality of the concept presented above in FIG. 2—i.e. that cancer cells release exosomes with membrane HSP70 (HSP70-exosomes) while normal cells do not (or in negligible amounts)—the inventors have analyzed a large panel of normal and cancer cells:

Cancer cell lines analyzed are: melanoma (B16F10), colon cancer (SW480, HCT116, CT26), prostate (PC3), lymphona (EL4), cervix (HeLa) and breast cancer (MCF7).

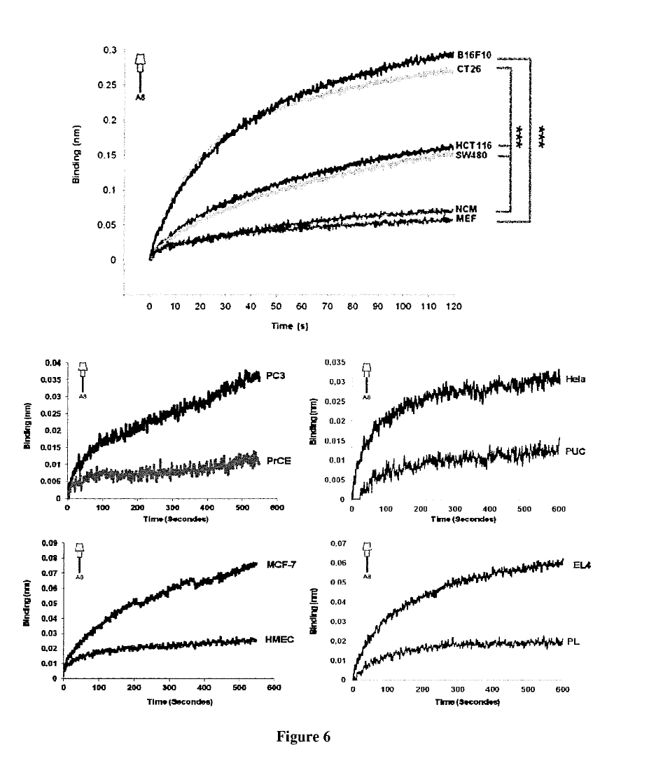

Normal (non-cancerous) cells analyzed besides the MEF are: primary normal colon mucosa cells (NCM), uterine cells (PUC), prostate epithelial cells (PrEC), breast epithelial cells (HMEC) and primary lymphocytes (PL) The inventors have used biolayer interference (BLI) protocol using an Octet instrument and HSP70 peptide aptamer A8 as a high affinity ligand to capture HSP70-exosomes from the cells' supernatants. The results obtained with all the cells analyzed clearly show that all cancer cells released a high amount of HSP70-exosomes as compared with their normal (non-cancerous) counterparts where hardly any release of HSP70-exosomes could be detected (FIG. 6).

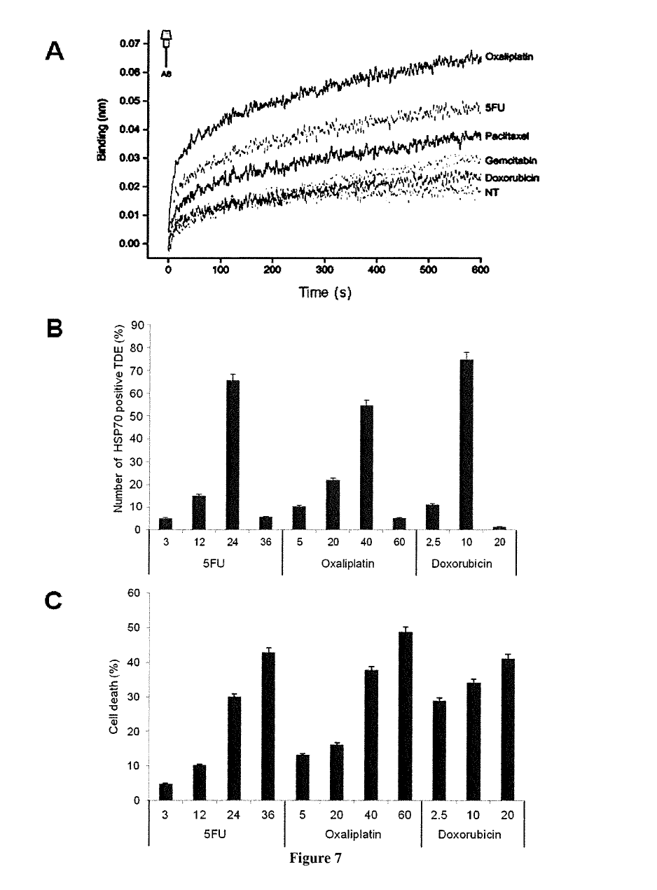

In FIG. 5, the inventors show the ability in vitro and in vivo of the anti-cancer drug cisplatin to increase the number of HSP70-exosomes. To explore the ability of other anticancer drugs to induce HSP70-exosomes release from cancer cells, the inventors have tested 5-fluorouracil, paclitaxel, gemcitabine, oxaliplatin and doxorubicin. As shown in FIG. 7A, all drugs tested at toxic equivalent concentrations, to a greater (oxaliplatin, 5-fluorouracil) or lesser (doxorubicin) extent, induce an increase in the amount of HSP70-exosomes. A dose-response release of HSP70-exosomes for cisplatin, 5-fluorouracil and doxorubicin is shown in FIG. 7B. The inventors observed for the 3 drugs that the amount of HSP70-exosomes released increased with drug concentration, but only for concentrations inducing a relatively low amount of cell death. At higher doses inducing more than 40% cell death (FIG. 7C), the number of exosomes drastically decreased, suggesting that increased HSP70-exosomes is not just due to cell death and release but probably is part of the cancer cell protective stress response involving the induction of HSP70 expression.

HSP70 Peptide Aptamer A8 Restores Anticancer Immune Response by Targeting MDSC.

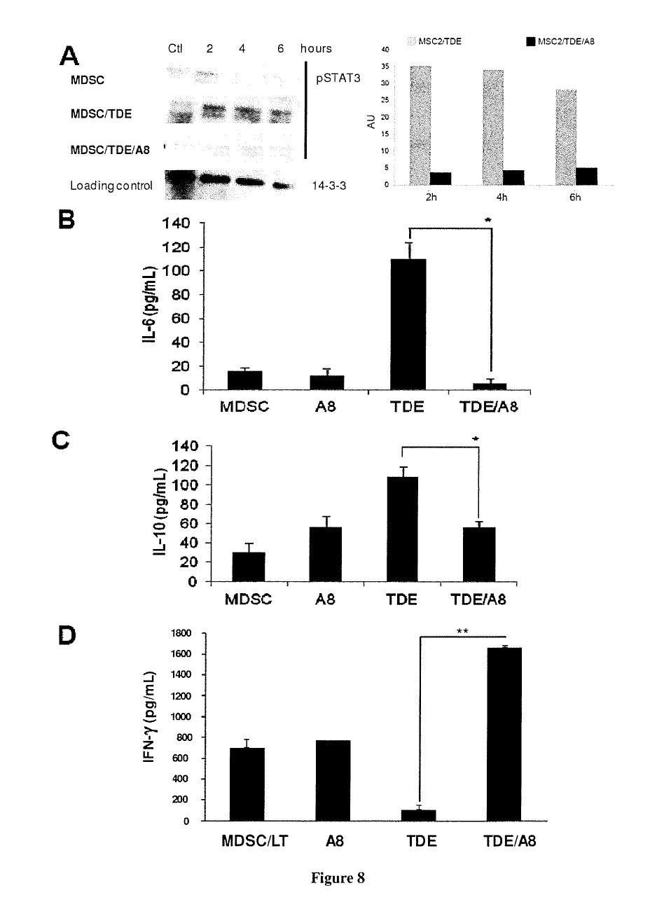

The inventors have strengthened the demonstration that A8 blocks the effect of HSP70-exosomes activating MDSC by using primary MDSC cells isolated from the spleen of mice. Further, the inventors have used four different read-out of MDSC activity: IL6 release, STAT-3 phosphorylation, IL10 release and their ability to induce IFNgamma release by T cells. For the last, the inventors have tested the ability of primary cultures of MDSC to induce IFNgamma secretion by naïf T-cells. As shown in FIG. 8, A8 blocks all MDSC activation markers.

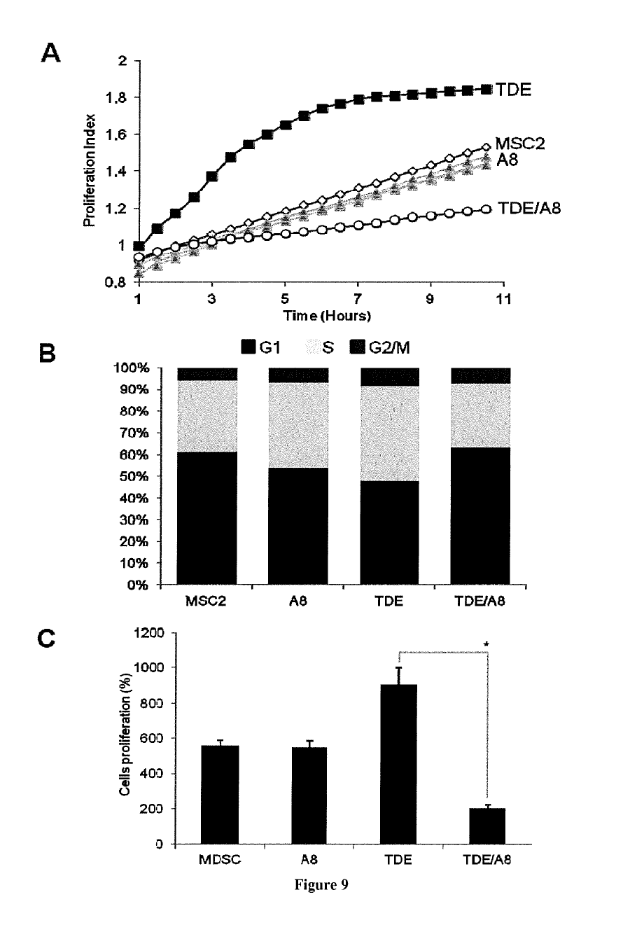

The inventors demonstrate that A8 not only blocked TDE-induced MDSC activation but also proliferation as shown in cultures of both MSC2 and primary MDSC (FIG. 9). The effect of A8 in the number of MDSC is probably a consequence of its effect blocking their activation and thereby the IL-6-dependent proliferation loop (Chang Q et al, Neoplasia 2013).

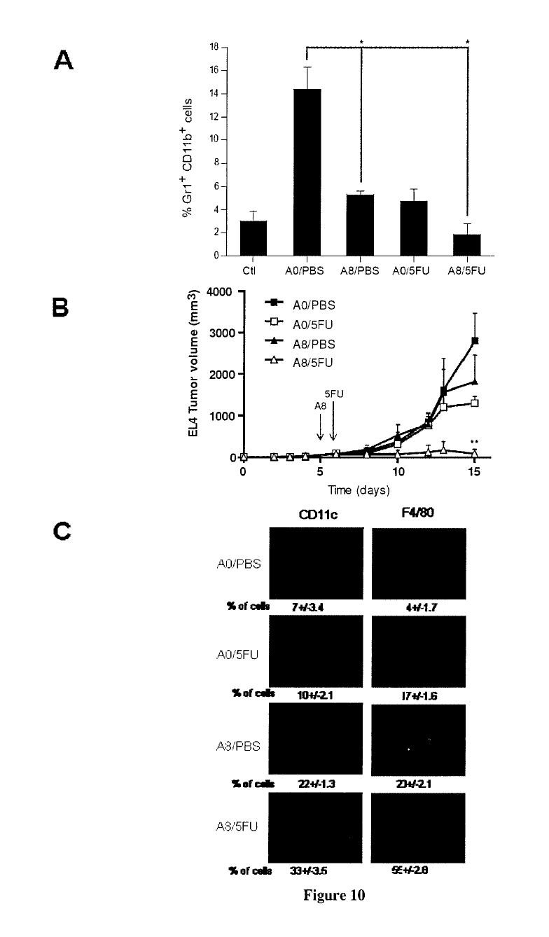

To confirm A8 in vivo results shown in FIG. 5, the inventors have used another syngeneic model: lymphoma EL4 tumors developed in C57BL6 mice. The results obtained were similar to that obtained with the B16F10 mice melanoma model. A8 induced in EL4 model: a) a decrease in the number of MDSC in the spleen; b) a decrease in the size of the tumors mainly when added in combination to 5FU, the drug more adapted to this model; and c) the tumors were strongly infiltrated by different immune cells attesting of the mice anti-cancer immune response (FIG. 10).

### REFERENCES

Throughout this application, various references describe the state of the art to which this invention pertains. The disclosures of these references are hereby incorporated by reference into the present disclosure.
1. Birtle A (2013) Maximizing survival in metastatic castrate-resistant prostate cancer: a clinical viewpoint. Expert Rev Anticancer Ther 13: 89-99. doi:10.1586/era.12.160.
2. Pasini F, Fraccon A P, D E Manzoni G (2011) The role of chemotherapy in metastatic gastric cancer. Anticancer Res 31: 3543-3554.
3. Siegel R, DeSantis C, Virgo K, Stein K, Mariotto A, et al. (n.d.) Cancer treatment and survivorship statistics, 2012. CA Cancer J Clin 62: 220-241. doi:10.3322/caac.21149.
4. Kigawa J (2013) New strategy for overcoming resistance to chemotherapy of ovarian cancer. Yonago Acta Med 56: 43-50.
5. Liu Y, Wang L, Predina J, Han R, Beier U H, et al. (2013) Inhibition of p300 impairs Foxp3+ T regulatory cell function and promotes antitumor immunity. Nat Med: 1-7. doi:10.1038/nm.3286.
6. Chhabra A, Mukherji B (2013) Death Receptor-Independent Activation-Induced Cell Death in Human Melanoma Antigen-Specific MHC Class I-Restricted TCR-Engineered CD4 T Cells. J Immunol. doi:10.4049/jimmunol.1202350.
7. Galdiero M R, Bonavita E, Barajon I, Garlanda C, Mantovani A, et al. (2013) Tumor associated macrophages and neutrophils in cancer. Immunobiology. doi:10.1016/j.imbio.2013.06.003.
8. Khaled Y S, Ammori B J, Elkord E (2013) Myeloid-derived suppressor cells in cancer: recent progress and prospects. Immunol Cell Biol. doi:10.1038/icb.2013.29.
9. Nagaraj S, Collazo M, Corzo C A, Youn J-I, Ortiz M, et al. (2009) Regulatory myeloid suppressor cells in health and disease. Cancer Res 69: 7503-7506. doi:10.1158/0008-5472.CAN-09-2152.
10. Serafini P, Borrello I, Bronte V (2006) Myeloid suppressor cells in cancer: recruitment, phenotype, properties, and mechanisms of immune suppression. Semin Cancer Biol 16: 53-65. doi:10.1016/j.semcancer.2005.07.005.
11. Serafini P, De Santo C, Mango I, Cingarlini S, Dolcetti L, et al. (2004) Derangement of immune responses by myeloid suppressor cells. Cancer Immunol Immunother 53: 64-72. doi:10.1007/s00262-003-0443-2.
12. Chalmin F, Ladoire S, Mignot G, Vincent J, Bruchard M, et al. (2010) Membrane-associated Hsp72 from tumor-derived exosomes mediates STAT3-dependent immunosuppressive function of mouse and human myeloid-derived suppressor cells. 120: 457-471. doi:10.1172/JC140483DS1.
13. Bobrie A, Théry C (2013) Exosomes and communication between tumours and the immune system: are all exosomes equal? Biochem Soc Trans 41: 263-267. doi:10.1042/BST20120245.
14. Ostrowski M, Carmo N B, Krumeich S, Fanget I, Raposo G, et al. (2010) Rab27a and Rab27b control different steps of the exosome secretion pathway. Nat Cell Biol 12: 19-30; sup pp 1-13. doi:10.1038/ncb2000.
15. McLellan A D (2009) Exosome release by primary B cells. Crit Rev Immunol 29: 203-217.
16. Knight A M (2008) Regulated release of B cell-derived exosomes: do differences in exosome release provide insight into different APC function for B cells and DC? Eur J Immunol 38: 1186-1189. doi:10.1002/eji.200838374.
17. Kapsogeorgou E K, Abu-Helu R F, Moutsopoulos H M, Manoussakis M N (2005) Salivary gland epithelial cell exosomes: A source of autoantigenic ribonucleoproteins. Arthritis Rheum 52: 1517-1521. doi:10.1002/art.21005.
18. Théry C, Ostrowski M, Segura E (2009) Membrane vesicles as conveyors of immune responses. Nat Rev Immunol 9: 581-593. doi:10.1038/nri2567.
19. Valadi H, Ekström K, Bossios A, Sjöstrand M, Lee J J, et al. (2007) Exosome-mediated transfer of mRNAs and microRNAs is a novel mechanism of genetic exchange between cells. Nat Cell Biol 9: 654-659. doi:10.1038/ncb1596.
20. Peinado H, Ale\check{c}ković M, Lavotshkin S, Matei I, Costa-Silva B, et al. (2012) Melanoma exosomes educate bone marrow progenitor cells toward a pro-metastatic phenotype through MET. Nat Med 18: 883-891. Available: http://www.ncbi.nlm.nih.gov/pubmed/22635005. Accessed 1 Nov. 2012.
21. Bobrie A, Colombo M, Raposo G, Théry C (2011) Exosome secretion: molecular mechanisms and roles in immune responses. Traffic 12: 1659-1668. doi:10.1111/j.1600-0854.2011.01225.x.
22. Lv L-H, Wan Y-L, Lin Y, Zhang W, Yang M, et al. (2012) Anticancer drugs cause release of exosomes with heat shock proteins from human hepatocellular carcinoma cells that elicit effective natural killer cell antitumor responses in vitro. J Biol Chem 287: 15874-15885. doi:10.1074/jbc.M112.340588.
23. Balaburski G M, Leu J I-J, Beeharry N, Hayik S, Andrake M D, et al. (2013) A modified HSP70 inhibitor shows broad activity as an anticancer agent. Mol Cancer Res. doi:10.1158/1541-7786.MCR-12-0547-T.
24. Lanneau D, Brunet M, Frisan E, Solary E, Fontenay M, et al. (2008) Heat shock proteins: essential proteins for apoptosis regulation. J Cell Mol Med 12: 743-761. doi:10.1111/j.1582-4934.2008.00273.x.
25. Schmitt E, Parcellier A, Gurbuxani S, Cande C, Hammann A, et al. (2003) Chemosensitization by a Non-apoptogenic Heat Shock Protein 70-Binding Apoptosis-Inducing Factor Mutant Chemosensitization by a Non-apoptogenic Heat Shock Protein 70-Binding Apoptosis-Inducing Factor Mutant: 8233-8240.
26. Rérole A-L, Gobbo J, De Thonel A, Schmitt E, Pais de Barros J P, et al. (2011) Peptides and aptamers targeting HSP70: a novel approach for anticancer chemotherapy. Cancer Res 71: 484-495. doi:10.1158/0008-5472.CAN-10-1443.
27. Jego G, Hazoumé A, Seigneuric R, Garrido C (2013) Targeting heat shock proteins in cancer. Cancer Lett 332: 275-285. doi:10.1016/j.canlet.2010.10.014.
28. Rappa F, Farina F, Zummo G, David S, Campanella C, et al. (2012) HSP-molecular chaperones in cancer biogenesis and tumor therapy: an overview. Anticancer Res 32: 5139-5150.
29. Multhoff G, Botzler C, Wiesnet M, Muller E, Meier T, et al. (1995) A Stress-inducible 72-kDa Heat-Shock Protein (HSP72) is expressed on the surface of human tumor cells. 279: 272-279.
30. Multhoff G (2007) Heat shock protein 70 (Hsp70): Membrane location, export and immunological relevance. 43: 229-237. doi:10.1016/j.ymeth.2007.06.006.
31. Stangl S, Gehrmann M, Riegger J, Kuhs K, Riederer I, et al. (2011) Targeting membrane heat-shock protein 70 (Hsp70) on tumors by cmHsp70.1 antibody. Proc Natl Acad Sci USA 108: 733-738. doi:10.1073/pnas.1016065108.
32. Baines I C, Colas P (2006) Peptide aptamers as guides for small-molecule drug discovery. Drug Discov Today 11: 334-341. doi:10.1016/j.drudis.2006.02.007.
33. Seigneuric R, Gobbo J, Colas P, Garrido C (2011) Targeting cancer with peptide aptamers. Oncotarget 2: 557-561.
34. Stangl S, Themelis G, Friedrich L, Ntziachristos V, Sarantopoulos A, et al. (2011) Detection of irradiation-induced, membrane heat shock protein 70 (Hsp70) in mouse tumors using Hsp70 Fab fragment. Radiother Oncol 99: 313-316. doi:10.1016/j.radonc.2011.05.051.
35. Kapanadze T, Gamrekelashvili J, Ma C, Chan C, Zhao F, et al. (2013) Regulation of accumulation and function of myeloid derived suppressor cells in different murine models of hepatocellular carcinoma. J Hepatol. doi:10.1016/j.jhep.2013.06.010.
36. Xiang X, Liu Y, Zhuang X, Zhang S, Michalek S, et al. (2010) TLR2-mediated expansion of MDSCs is dependent on the source of tumor exosomes. Am J Pathol 177: 1606-1610. doi:10.2353/ajpath.2010.100245.
37. Kusmartsev S, Cheng F, Yu B, Nefedova Y, Sotomayor E, et al. (2003) All-trans-retinoic acid eliminates immature myeloid cells from tumor-bearing mice and improves the effect of vaccination. Cancer Res 63: 4441-4449.
38. Chang Q, Bournazou E, Sansone P, Berishaj M, Gao S P, et al. (2013) The IL-6/JAK/Stat3 Feed-Forward Loop Drives Tumorigenesis. 15: 848-862. doi:10.1593/neo.13706.
39. Lindau D, Gielen P, Kroesen M, Wesseling P, Adema G J (2013) The immunosuppressive tumour network: myeloid-derived suppressor cells, regulatory T cells and natural killer T cells. Immunology 138: 105-115. doi:10.1111/imm.12036.
40. Hanahan D, Weinberg R A, Francisco S (2000) The Hallmarks of Cancer Review University of California at San Francisco. 100: 57-70.
41. Andre F, Dieci M V, Dubsky P, Sotiriou C, Curigliano G, et al. (2013) Molecular pathways: involvement of immune pathways in the therapeutic response and outcome in breast cancer. Clin Cancer Res 19: 28-33. doi:10.1158/1078-0432.CCR-11-2701.
42. Galluzzi L, Senovilla L, Zitvogel L, Kroemer G (2012) The secret ally: immunostimulation by anticancer drugs. Nat Rev Drug Discov 11: 215-233. doi:10.1038/nrd3626.
43. Martins I, Kepp O, Schlemmer F, Adjemian S, Tailler M, et al. (2011) Restoration of the immunogenicity of cisplatin-induced cancer cell death by endoplasmic reticulum stress. Oncogene 30: 1147-1158. doi:10.1038/onc.2010.500.
44. Obeid M, Tesniere A, Ghiringhelli F, Fimia G M, Apetoh L, et al. (2007) Calreticulin exposure dictates the immunogenicity of cancer cell death. Nat Med 13: 54-61. doi:10.1038/nm1523.
45. Rérole A-L, Jego G, Garrido C (2011) Hsp70: anti-apoptotic and tumorigenic protein. Methods Mol Biol 787: 205-230. doi:10.1007/978-1-61779-295-3_16.
46. Coles D J, Rolfe B E, Boase N R B, Veedu R N, Thurecht K J (2013) Aptamer-targeted hyperbranched polymers: towards greater specificity for tumours in vivo. Chem Commun (Camb) 49: 3836-3838. doi:10.1039/c3cc00127j.
47. Chang Q, Bournazou E, Sansone P, et al. The IL-6/JAK/Stat3 Feed-Forward Loop Drives Tumorigenesis. Neoplasia. 2013; 15(7):848-862. doi:10.1593/neo.13706.

1. An in vitro method for i) diagnosing cancer in a subject, or for ii) determining responsiveness of a patient to a treatment with a chemotherapeutic agent, comprising
determining a level of Heat Shock Protein 70 (HSP70)-expressing exosomes in a bodily fluid sample obtained from said subject; and, based on the level of Heat Shock Protein 70 (HSP70)-expressing exosomes determined in determining step, respectively:
diagnosing cancer in said subject, or
determining the responsiveness of the patient to the treatment with the chemotherapeutic agent.

2. The method according to claim 1, further comprising a step of comparing said level of HSP70-expressing exosomes with reference values from healthy subjects, wherein detecting an increase in the level of HSP70-expressing exosomes compared to the reference values indicates that the subject has, or is at risk of having cancer.

3. (canceled)

4. The method according to claim 1, further comprising a step of comparing the level of HSP70-expressing exosomes after treatment with the chemotherapeutic agent with reference values from the patient before treatment with the chemotherapeutic agent, wherein detecting a decrease in the level of HSP70-expressing exosomes compared to the reference values indicates that the patient will be or is a responder to said treatment with a chemotherapeutic agent.

5. The method according to claim 1, wherein the step of determining the level of HSP70-expressing exosomes is carried out by using a HSP70-binding agent that binds to an extracellular localized epitope of membrane-bound HSP70 on said exosomes.

6. The method according to claim 5 wherein said HSP70-binding agent is a peptide of SEQ ID NO: 2 or a function-conservative variant thereof.

7. The method according to claim 1, wherein the biological sample is a bodily fluid sample.

8. The method according to claim 1, wherein the cancer is selected from the group consisting of lung, colon, breast, ovary head and neck, stomach, pancreas carcinomas, malignant melanoma, and hematological cancers

9. The method according to claim 1, wherein said chemotherapeutic agent is selected from the group consisting of a microtubule active agent, an anti-neoplastic anti-metabolite, a platin compound and a topoisomerase II inhibitor.

10-12. (canceled)

13. The method of claim 17, wherein said chemotherapeutic agent is selected from the group consisting of a microtubule active agent, an anti-neoplastic anti-metabolite, a platin compound and a topoisomerase II inhibitor.

14. The method of claim 17, wherein said HSP70-binding agent is a peptide of SEQ ID NO: 2 or a function-conservative variant thereof.

15. An in vitro method for determining whether a patient will benefit from a treatment with an inhibitor of the interaction between HSP70 and TLR2, comprising a step of determining a level of HSP70-expressing exosomes in a bodily fluid sample obtained from said patient, and comparing said level of HSP70-expressing exosomes with reference values from healthy subjects, wherein detecting an increase in the level of HSP70-expressing exosomes compared to the reference values indicates that the patient will benefit from a treatment with an inhibitor of interaction between HSP70 and TLR2.

16. The method of claim 7, wherein the bodily fluid sample is a blood sample or a urine sample.

17. A method for inhibiting activation of myeloid-derived suppressor cells (MDSC) in a subject in need thereof in order to
i) restore or enhance an anti-tumor immune response;
ii) inhibit or reduce HSP70-expressing exosome-mediated tumor resistance against a chemotherapeutic agent; or
iii) restore or enhance efficacy of a chemotherapeutic agent;
comprising administering to said subject an inhibitor of interaction between HSP70 and Toll-like receptor 2 (TLR2), wherein said inhibitor is a HSP70 binding agent that specifically binds to an epitope of HSP70 that is localized extracellularly on exosomes.

18. The method of claim 15, wherein the inhibitor of interaction between HSP70 and Toll-like receptor 2 (TLR2) is a peptide of SEQ ID NO: 2.
